(19) United States
(12) Patent Application Publication
(10) Pub. No.: US 2024/0008915 A1
(43) Pub. Date: Jan. 11, 2024

(54) HEART VALVE ABLATION CATHETER

(71) Applicant: Bio ReFine Ltd., Tel-Aviv (IL)

(72) Inventors: Dvir KEREN, Tel Aviv (IL); Boaz SHENHAV, Tel-Aviv (IL)

(73) Assignee: Bio ReFine Ltd., Tel-Aviv (IL)

(21) Appl. No.: 18/035,782

(22) PCT Filed: May 4, 2021
(86) PCT No.: PCT/IL2021/050512

§ 371 Date: May 8, 2023

Related U.S. Application Data
(60) Provisional application No. 63/111,033, filed on Nov. 8, 2020.

(51) Int. Cl. A61B 18/14

(52) U.S. Cl. CPC A61B 18/1477; A61B 2018/00369

(57) ABSTRACT

Cardiac annuloplasty methods and devices, based on delivery of tissue-ablating energy to a heart valve annulus, thereby inducing reduction of the valve annulus perimeter. In some embodiments, the reduction is induced by shrinkage of tissue in response to ablation, potentially analogous to tissue shrinkage responsible for pulmonary vein stenosis induced by cardiac ablation to treat atrial fibrillation. In some embodiments, annulus tissue is deformed before ablation energy is applied. This potentially results in plastic deformation apart from tissue shrinkage. Deformation is optionally performed using electrodes that also operate as sharp-tipped jaws of a pliers. They are inserted to tissue in a wider-spaced configuration, reduced in distance to a narrower-spaced tissue-squeezing configuration, and operated to ablate the squeezed tissue. Upon removal of the electrodes, the squeezed tissue retains a new shape as a result of ablation-induced plastic deformation.

### RELATED APPLICATIONS

This application claims the benefit of priority under 35 USC § 119(e) of U.S. Provisional Patent Application No. 63/111,033 filed Nov. 8, 2020; the contents of which are incorporated herein by reference in their entirety.

### FIELD AND BACKGROUND OF THE INVENTION

The present invention, in some embodiments thereof, relates to the field of structural heart disease and more particularly, but not exclusively, to heart valve annuloplasty.

Patients who suffer from insufficient heart valve function (for example, of the mitral valve) may undergo implantation of an annuloplasty ring, sutured to the heart valve's fibrous annulus tissue. The aim is to shrink and/or stabilize the valve's perimeter. The procedure may be carried out as an open heart surgery, or in with some devices via an intravascular (transcatheter) approach.

As the valve's perimeter is shrunk, the valve's leaves get closer, therefore achieving a better sealing (coaptation) to reduce or eliminate valve regurgitation.

### SUMMARY OF THE INVENTION

According to an aspect of some embodiments of the present disclosure, there is provided a method of performing a cardiac annuloplasty procedure, including delivering energy to a perimeter of a heart valve annulus, in an amount sufficient to structurally disrupt tissue and induce shrinkage of the valve annulus perimeter to reduce regurgitation through the heart valve.

According to some embodiments of the present disclosure, the structural disruption of tissue includes changes to the fibrotic structure of the tissue.

According to some embodiments of the present disclosure, the energy disrupts the tissue while the heart valve annulus tissue in its mechanically deformed condition.

According to some embodiments of the present disclosure, the mechanically deforming includes compressing on the tissue.

According to some embodiments of the present disclosure, the compressing the tissue includes piercing the tissue with at least one sharpened element, and manipulating the sharpened element to exert the compression.

According to some embodiments of the present disclosure, the at least one sharpened element also includes an element used to deliver the structurally disruptive energy to the tissue.

According to some embodiments of the present disclosure, the element is an electrode.

According to some embodiments of the present disclosure, the electrode disrupts the tissue structure by transmitting RF energy into the tissue.

According to some embodiments of the present disclosure, the electrode disrupts structure of the tissue by induction of cellular death.

According to some embodiments of the present disclosure, the electrode disrupts structure of the tissue by coagulation.

According to some embodiments of the present disclosure, the compressing includes pinching the tissue between a plurality of the at least one sharpened element.

According to some embodiments of the present disclosure, the compressing includes exerting torsion on the tissue using the at least one sharpened element.

According to some embodiments of the present disclosure, the reduction of the valve annulus perimeter includes tissue shrinkage as a result of the delivery of tissue-ablating energy.

According to some embodiments of the present disclosure, the reduction of the valve annulus perimeter includes plastic deformation of tissue as a result of the delivery of tissue-ablating energy while the tissue is mechanically deformed.

According to some embodiments of the present disclosure, the delivering tissue-ablating energy includes delivery radiofrequency energy through the electrode into the pierced tissue.

According to some embodiments of the present disclosure, the delivering tissue-ablating energy includes delivery radiofrequency energy through the electrode into the contacted tissue.

According to some embodiments of the present disclosure, the tissue-ablating energy is provided by at least one of the group consisting of: radiofrequency energy; focused ultrasound energy; cryogenic cooling.

According to some embodiments of the present disclosure, the tissue ablated includes at least one of the group consisting of: fibrous tissue of the valve annulus; and tissue of the heart wall adjacent to the fibrous tissue of the valve annulus.

According to some embodiments of the present disclosure, the valve annulus is a valve annulus of mitral valve or a tricuspid valve.

According to some embodiments of the present disclosure, the method includes repeating the delivering of tissue-ablating energy at a plurality of sites along the perimeter of the heart valve annulus.

According to some embodiments of the present disclosure, the method includes: selecting a patient with an enlarged heart valve perimeter; planning a targeted reduction in heart valve annulus perimeter, including selection of locations along the heart valve annuls targeted for shrinkage; and performing the delivering energy at each of the selected locations.

According to some embodiments of the present disclosure, at least a portion of the shrinkage occurs during the cardiac annuloplasty procedure.

According to some embodiments of the present disclosure, at least a portion of the shrinkage occurs after the cardiac annuloplasty procedure.

According to an aspect of some embodiments of the present disclosure, there is provided a method of performing cardiac annuloplasty, including: piercing tissue along a perimeter of a heart valve with at least one electrode; applying mechanical force to the at least one electrode to deform the pierced tissue and reduce the perimeter of the heart valve annulus; delivering tissue-ablating energy through the electrode, thereby inducing plastic deformation of the deformed tissue; and releasing the mechanical force, leaving the heart valve annulus with a reduced perimeter.

According to some embodiments of the present disclosure, the applying mechanical force includes placing torsion on the pierced tissue.

According to some embodiments of the present disclosure, the applying mechanical force includes compressing the pierced tissue.

According to some embodiments of the present disclosure, the tissue-ablating energy is radiofrequency energy.

According to some embodiments of the present disclosure, the delivering tissue-ablating energy induces plastic deformation by coagulation.

According to some embodiments of the present disclosure, the reduced perimeter draws leaflets of the heart valve into positions that reduce regurgitation of the valve.

According to some embodiments of the present disclosure, the regurgitation reduction includes restoration of coaptation between the leaflets of the heart valve.

According to some embodiments of the present disclosure, the plastic deformation includes shrinkage of the deformed tissue.

According to an aspect of some embodiments of the present disclosure, there is provided a device for annuloplasty treatment, including: a catheter, sized for transvascular insertion to a heart chamber from a percutaneous incision to reach a heart valve annulus thereof; at least one tissue penetrating element at a distal end of the catheter; wherein the at least one penetrating element both moves relative to a body of the catheter, and acts to deliver tissue disrupting energy to penetrated tissue of the heart valve annulus.

According to some embodiments of the present disclosure, the at least one penetrating element includes a plurality of penetrating elements, adjustable in their relative distance while inserted to tissue of the heart valve annulus.

According to some embodiments of the present disclosure, each of the at least one tissue penetrating elements is an electrode electrically interconnected to a connection remaining outside the percutaneous incision when the catheter is inserted to the heart chamber.

According to some embodiments of the present disclosure, each of the plurality of penetrating elements operates as an ablation electrode.

According to some embodiments of the present disclosure, the penetrating elements are spaced to insert to the tissue at a relatively wider distance, and adjust to a narrower distance.

According to some embodiments of the present disclosure, the relative distance of the penetrating elements is adjusted by rotation of a gear.

According to some embodiments of the present disclosure, the gear is rotated by a control element leading to a proximal side of the catheter.

According to some embodiments of the present disclosure, the control element also acts to provide electrical interconnection between at least one of the tissue penetrating elements and a source of electrical power which remains outside of the percutaneous incision.

According to some embodiments of the present disclosure, the relative distance of the penetrating elements is adjusted by a temperature change of an actuator including a shape memory alloy.

According to some embodiments of the present disclosure, the shape memory allow is positioned so that the temperature change is induced by heating consequent to operation of the penetrating elements as electrodes.

According to some embodiments of the present disclosure, the shape memory alloy is shaped to move the penetrating elements from an initial distance to a relatively narrower distance when heated.

According to some embodiments of the present disclosure, the device includes a device reset, actuatable to restore the distance of the penetrating elements before the temperature change, while the device remains inserted to the heart chamber.

According to some embodiments of the present disclosure, the device includes an inner component terminating distally in an energy delivering segment, and housed within an outer tube; the outer tube being sized for insertion to the heart chamber from within a guiding catheter; wherein the outer tube is provided with a predetermined distal bend which it assumes when unconfined by the guiding catheter, and which straightens when the outer tube is withdrawn into the guiding catheter.

According to some embodiments of the present disclosure, the at least one penetrating element has a non-circular cross-section that engages with and induces torsion in tissue to which it is inserted, upon receiving torque exerted through the catheter.

According to some embodiments of the present disclosure, the device has: an inner component terminating distally in a tissue ablation segment, and housed within an outer tube; the outer tube being sized for insertion to the heart chamber from within a guiding catheter; wherein the outer tube is provided with a predetermined distal bend which it assumes when unconfined by the guiding catheter, and which straightens when the outer tube is withdrawn into the guiding catheter.

According to some embodiments of the present disclosure, the non-circular cross-section includes a rectangular blade.

According to some embodiments of the present disclosure, the non-circular cross-section includes three or more blades radiating from a central axis.

Unless otherwise defined, all technical and/or scientific terms used herein have the same meaning as commonly understood by one of ordinary skill in the art to which the present disclosure pertains. Although methods and materials similar or equivalent to those described herein can be used in the practice or testing of embodiments of the present disclosure, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the present disclosure are herein described, by way of example only, with reference to the accompanying drawings. With specific reference now to the drawings in detail, it is stressed that the particulars shown are by way of example, and for purposes of illustrative discussion of embodiments of the present disclosure. In this regard, the description taken with the drawings makes apparent to those skilled in the art how embodiments of the present disclosure may be practiced.

In the drawings:

FIG. 1A-1B are flowcharts schematically describing a method of heart valve annulus treatment, according to some embodiments of the present disclosure;

FIG. 2A schematically illustrates an electrical monopolar ablation system, according to some embodiments of the present disclosure;

FIG. 2B schematically illustrates a bipolar ablation system, according to some embodiments of the present disclosure;

FIGS. 3, 4 and 5 schematically illustrate distal elements of an ablating catheter (optionally an example of an RF ablating catheter, or an ablating catheter using another ablation energy type; for example a cryoablation catheter or a focused ultrasound ablation catheter), related in particular to steering, according to some embodiments of the present disclosure;

FIGS. 6 and 7 schematically illustrate steering of ablating catheter within a heart chamber (left atrium), according to some embodiments of the present disclosure;

FIGS. 8A-8D schematically illustrate additional configurations of ablating catheter, out of the variety which enables him to ablate any location along the annulus, according to some embodiments of the present disclosure;

FIGS. 9, 10, 11, 12, 13, 14, 15, 16, 17, 18, 19 and 20 schematically illustrate alternative designs of inner component, for use with an RF ablating system, according to some embodiments of the present disclosure;

FIGS. 21A-21B, 22, 23, 24A-24B and 25 schematically illustrate a method of shaping tissue by ablation while the tissue is in compression or traction, according to some embodiments of the present disclosure;

FIGS. 26 and 27A-27B schematically illustrate electrode pliers, according to some embodiments of the present disclosure;

FIGS. 28A-28C schematically illustrate a covered catheter tip casing encasing the mechanism of FIGS. 27A-27C, according to some embodiments of the present disclosure;

FIG. 29 schematically illustrates positioning via an endovascular approach of the distal portion of an ablation catheter used for annuloplasty of a mitral valve, according to some embodiments of the present disclosure;

FIG. 30 demonstrates an optional proximal side of a catheter, according to some embodiments of the present disclosure;

FIGS. 31A-31E schematically illustrate different constructed layers of an adjustable-width ablation catheter, according to some embodiments of the present disclosure;

FIG. 32 illustrates an unfolded view of a self-interlocking pattern cut to provide flexibility to tube and/or tube, according to some embodiments of the present disclosure;

FIG. 33 schematically represents an ablation electrode configuration which inserts to tissue, twists, then ablates, according to some embodiments of the present disclosure;

FIGS. 34A-34D illustrate a twist-and-ablate method of valve perimeter reduction, according to some embodiments of the present disclosure;

FIGS. 35A-35B schematically illustrate configurations of an electrode assembly comprising two needle electrodes interconnected by a loop spring, according to some embodiments of the present disclosure;

FIGS. 36A-36E schematically illustrate construction of a mechanically actuated electrode assembly comprising two needle electrodes interconnected by a loop spring, according to some embodiments of the present disclosure; and

FIGS. 37A-37C schematically illustrate operation of the mechanically actuated electrode assembly of FIGS. 36A-36E, according to some embodiments of the present disclosure.

### DESCRIPTION OF SPECIFIC EMBODIMENTS OF THE INVENTION

The present invention, in some embodiments thereof, relates to the field of structural heart disease and more particularly, but not exclusively, to heart valve annuloplasty.

### Overview

An aspect of some embodiments of the present disclosure relates to valve annuloplasty performed using tissue shrinkage and/or remodeling induced by energy applied to the region of the valve's annular ring.

Currently a gold standard of care to treat atrial fibrillation is the use of RF energy to ablate regions along the left atrial wall around the pulmonary veins. A reported side effect of this procedure is pulmonary vein stenosis (PVS).

PVS may also be an outcome of ablation procedures performed using other methods such as cryoablation (e.g., as reported by J. Matsuda et al., J Cardiovasc Electrophysiol. 2017 March; 28(3):298-303. Pulmonary Vein Stenosis After Second-Generation Cryoballoon Ablation).

PVS is attributed to shrinkage of the pulmonary veins induced by shrinkage of ablated tissue areas. A physiological mechanism which creates stenosis in pulmonary veins due to ablation is attributed to scarring of connective tissue surrounding the pulmonary veins, for example as described in Pulmonary Vein Stenosis After Catheter Ablation, Electroporation Versus Radiofrequency by Vincent JAM et al., Circ Arrhythm Electrophysiol. 2014 August; 7(4):734-8.

The inventors describe herein an endovascular approach using the phenomenon of tissue shrinkage induced by applying structurally disruptive energy to treat heart valve leakage. Leakage is characterized by failure of the heart valve's leaflets to coapt—they do not close fully in response to back-pressure. This allows blood flow regurgitation, and impairs the efficiency of pumping by the heart.

In some embodiments of the present disclosure, tissue on the perimeter of the valve annulus is remodeled by the application of structurally disruptive energy. In some embodiments, this comprises energy sufficient to ablate tissue. Ablation may lesion the valve annulus tissue directly (that is, ablation lesions fibrous tissue of the valve annulus), and/or tissue nearby; for example, the atrial wall above the mitral or tricuspid valve.

The ablation induces shrinkage—and a corresponding reduction in the overall valve annulus perimeter. This potentially brings leaflets of a regurgitating heart valve into coaptation; or if coaptation is not achieved, may reduce the severity of the regurgitation by reducing the remaining gap between them in their most-closed state. In some cases, an original loss of normal valve leaflet coaptation was itself caused by reshaping (lengthening) the valve annulus. Accordingly, a treatment that shrinks the valve annulus may return the heart valves into their original relationship with one another.

Herein, reference to “ablation” of tissue refers to the delivery of structurally disruptive energy to the tissue which at least induces cellular death in the tissue while generally retaining—although also modifying—the integrity of the tissue's connective structure. Furthermore, within the context of embodiments described herein, the ablation performed has at least one of the following two:
The ablated tissue shrinks.
The ablated tissue undergoes plastic remodeling to a shape influenced by mechanical forces imposed on the tissue during and/or after the ablation.

Without commitment to a particular theory, these effects may result, for example, from loss of cellular structures, from relaxation of internal stresses on connective fibers, from effects of denaturation (coagulation) on tissue structures that persist after ablation, and/or from effects of healing processes which occur post-ablation.

Shrinkage may comprise effects which occur immediately or almost immediately (e.g., due to losses of fluid or shrinkage of cellular components), and slower effects due, e.g., to induced atrophy and/or processes of healing.

In some embodiments, application of structurally disruptive energy is optionally sub-ablative. For example, the fibrotic structure of tissue may be made more malleable by heating, and/or by adjusting its pH by the passage of an electrolyzing current. The structural disruption which produces this malleability may be induced concurrently with or separately from tissue shrinkage.

Effects of plastic remodeling under mechanical force described for embodiments herein are generally acute; that is, they occur during the application of structurally disruptive energy or within a brief period thereafter, while a procedure is underway that uses a tool to apply the mechanical force. Without commitment to a particular theory, these acute effects may be understood as influenced by the disorganizing effects of coagulation acting to relieve stresses and/or strains in tissue deformed by external mechanical forces. This effectively gives the tissue a new “preferred shape” in its coagulated state, even after the external mechanical forces are removed (i.e., it is plastically deformed). It is not excluded that there may be non-coagulating mechanisms influencing plastic deformation when applying structurally disruptive energy to tissue deformed by external forces. For example, a mechanism has been proposed by which electrolysis of water in a tissue region results in the production of free protons which in turn temporarily affect self-binding of the collagen matrix so that it become more malleable.

Two main types of ablation performed on heart tissue for treating atrial fibrillation are thermal ablation, e.g., using radiofrequency (RF) energy or ultrasound energy; and cryoablation. Both types of ablation have been associated with pulmonary vein stenosis. However, there may be differences in tissue remodeling effects as a result of differences in the two mechanisms. For example, thermal ablation effects include coagulation acting directly on structural cellular components, while cryoablation's main effects disrupt cellular organization and processes leading to downstream degeneration of structural cellular components, potentially under biological control. Electroporation is another cellular ablation mechanism which is primarily disruptive in its initial effects rather than denaturing (coagulating). Electrolysis of tissue water has also been proposed as a mechanism for disrupting the collagen matrix by partially acidifying it.

In some embodiments, annuloplasty performed by structural disruption of tissue reduces valve perimeter by up to about 5-10%. Reshaping of the valve annulus may be targeted to sites at any selected portion of the valve annulus perimeter; for example, disrupting tissue at approximately evenly spaced locations, or alternatively at locations grouped in one or more particular regions around the perimeter.

An aspect of some embodiments of the present disclosure relates to methods and devices for mechanically deforming tissue while also applying structurally disruptive energy to make tissue shrink and/or become more malleable. In some embodiments, the tissue is deformed by application of mechanical force. In some embodiments, the deformation is used to influence tissue remodeling effects of ablation, or sub-ablative application of structurally disruptive energy, to perform annuloplasty.

The effects of mechanical tissue deformation may be distinguished from those of structurally disruptive energy delivery, insofar as the mechanical tissue deformation (if applied without additional structurally disruptive energy) reverses when a mechanical force which induces it is removed. In other words, the mechanical tissue deformation alone is elastic; while the application of structurally disruptive energy “plasticizes” tissue, making it malleable by the deformation into a new, non-elastically reversing shape, and/or directly induces plastic deformation in the tissue. Reference herein to “disruption” of tissue refers to non-elastic, structural disruption of tissue with or without cell death. It should be understood that the disruption referred to is a partial disruption which modifies but maintains the overall structural integrity of the tissue.

Modalities of supplying structurally disruptive energy to tissue (such as RF ablation and/or the application of electrical current) may induce remodeling of tissue deformed by mechanical force into a new shape that persists when a mechanical force causing tissue deformation is removed. This type of plastic deformation is distinct from plastic deformation due to shrinkage of ablated tissue, and both effects may occur.

In some embodiments, mechanical force is applied by compressing tissue between a plurality of laterally separated elements. Each of these is also referred to herein as a “jaw”; they are also referred to herein as working together as a tissue pliers. The jaws are sized for the manipulation of the valve annulus, for example, having cross sections with a maximum width less than 0.2 mm, 0.4 mm, 0.8 mm, or 1 mm (for example, a 0.4 mm by 0.4 mm cross section), and about 1-10 mm in length, for example, 2 mm, 3 mm, or 4 mm. Maximum distance between the jaws may be, for example, in the range of about 2-10 mm, for example, 2 mm or 4 mm.

Jaws of the tissue pliers may be applied to the tissue surface, or they may pierce the tissue surface. When the jaws move toward each other, tissue is compressed, resulting in a deformation of its shape. Energy applied to the tissue in this state (e.g., in the form of heating, cooling, and/or electrical energy) may tend to relax internal forces of that deformed shape as tissue components are altered, e.g., coagulated and/or disassociated.

The jaws of the tissue pliers are optionally actuated by rotational movement commanded through a wire or shaft linking the tissue pliers through a catheter to a control actuator that remains outside the body (e.g., outside of a percutaneous incision through which the catheter was inserted). For example a rack-and-pinion arrangement may convert rotation of a pinion gear to linear movement of the jaws. Alternatively, the jaws are connected by ties to a central member rotated by the wire or shaft, and spring-loaded to remain separated until the central member turns, winding the ties shorter and bringing the jaws together.

In some embodiments, movement of the jaws is automatically induced by heating of the device during its operation to delivery energy to targeted tissue. This may be embodied, for example, using a shape memory alloy spring which is initialized in a first state (e.g, a jaws-separated state) while it is soft and below its transition temperature. The spring's preset shape in its superelastic state (above the alloy's transition temperature) is selected to drive the jaws to a closer position. After cooling, the device can be reset, for example, by using a reshaping device such as a wedge. Alternatively, there may be a second elastic member provided which is weaker than the shape memory alloy spring when above its transition temperature, but stronger when the shape memory allow spring is below its transition temperature.

Additionally or alternatively, in some embodiments, mechanical force is applied by inducing torsion in a tissue region (twisting it). The torsion may be applied by twisting a pliers engaged with the tissue (effectively using it as a wrench, with the pliers jaws doubling as wrench jaws). In another wrench-like configuration, a plurality of fixed jaws (since fixed, not used as pliers) re may be engaged with the tissue (e.g., by piercing it). By twisting these jaws around a common center, torsion is induced in the surrounding tissue.

Optionally, a single rod-like element is used as a wrench to exert torsion on surrounding tissue. This may comprise an element that pierces the tissue, and has a cross-sectional profile that such that some of its surfaces are forced against tissue when the single element is rotated. This can be a result of some parts of the cross-sectional profile having adjacent regions around the circumference at sharply exaggerated relative radially measured distances from the center of cross-sectional profile (that is, rapid transitions from wider to narrower). A narrow rectangular cross-section (e.g., blade-like) provides an example. Since tissue is somewhat elastic, there may be limits on blade torquing force before the aperture opens up enough to let the blade turn without dragging tissue with it. A related cross-sectional shape that may be used is a cross- or star-shape (e.g., having three, four or more blades radiating from a common central axis; a flat blade may be considered as having two blades each radiating from a common center). This may allow somewhat higher torque levels before slippage occurs. The maximum diameter of the portion of the device that inserts into tissue may be, for example, about 2-6 mm.

In some embodiments, delivery of structurally disruptive energy is performed using at least one of the same elements as comprise pliers and/or wrench jaws, for example, one or more of the jaws also operate as an ablation electrode.

The energy may be alternatively performed by an energy-delivering element (e.g, an electrode of an electrode probe, focused ultrasound probe, or cryoablation probe) which is placed on or in the tissue that has been deformed by a separate tool. This provides a potential advantage by allowing optional separation of the regions of highest applied force from those which receive the most energy. The region receiving the most structurally disruptive energy may also be the region which is most weakened by it, and the weakening may lead to unintended tearing.

Before explaining at least one embodiment of the present disclosure in detail, it is to be understood that the present disclosure is not necessarily limited in its application to the details of construction and the arrangement of the components and/or methods set forth in the following description and/or illustrated in the drawings. Features described in the current disclosure, including features of the invention, are capable of other embodiments or of being practiced or carried out in various ways.

### BACKGROUND

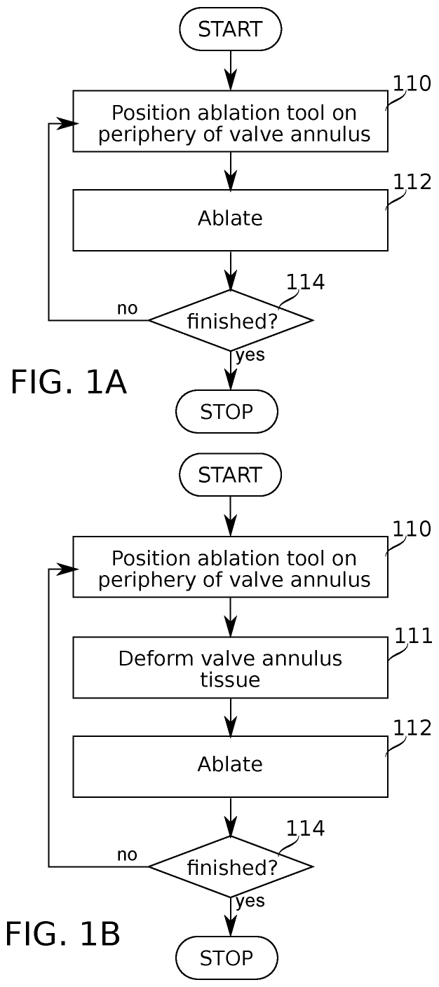

Reference is now made to FIG. 1A-1B, which are flowcharts schematically describing a method of heart valve annulus treatment, according to some embodiments of the present disclosure. The operations of the blocks of FIGS. 1A and 1B are substantially the same, with the exception that FIG. 1B adds block 111 for the operation of deforming valve annulus tissue.

At block 110, in some embodiments, an energy-delivery tool (e.g., an ablation electrode or other probe which can delivery structurally disruptive energy) is placed in position along a perimeter of a valve annulus. The position comprises contact with valve annulus tissue, and the contacted valve annulus tissue is tissue which is to be shrunk as part of an annuloplasty procedure which seeks to improve valve function by reducing overall valve annulus circumference.

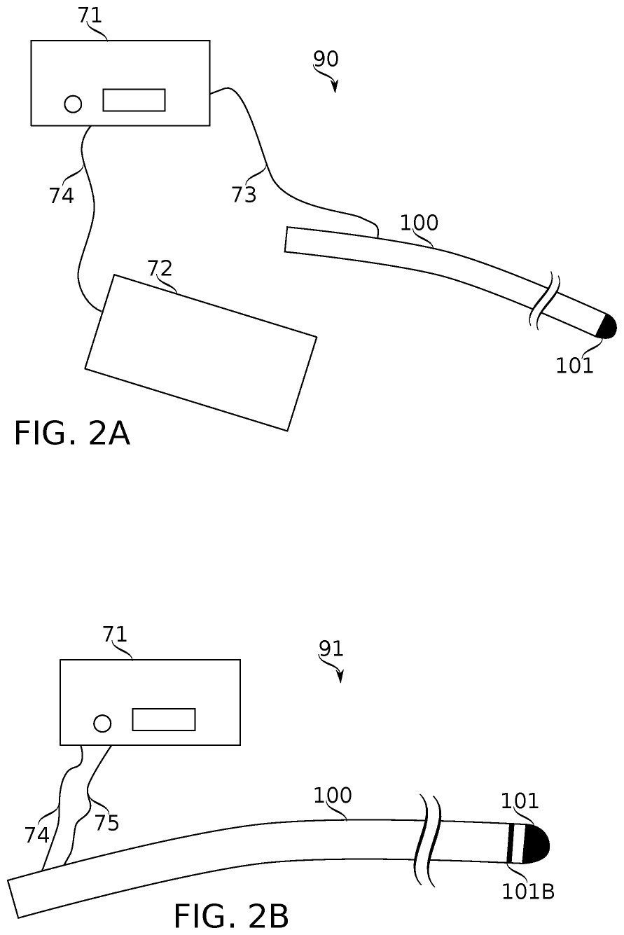

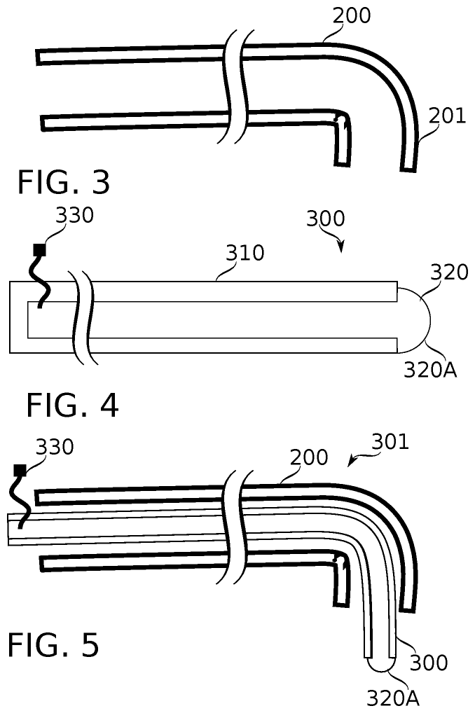

The energy-delivery tool may comprise, for example, an electrode configured to transmit radiofrequency (RF) energy into tissue, a focused ultrasound transducer, or a cryoablation probe. Examples of ablation systems are described, for example, in relation to FIGS. 2A-2B and 30. Other figures describe embodiments of probes used to deliver structurally disruptive energy. FIGS. 3-8D and 29 illustrate, in particular, probes provided with control degrees of freedom allowing accessing different parts of the perimeter of a valve annulus by contact with an energy-delivering portion (e.g., an electrode) of a catheter.

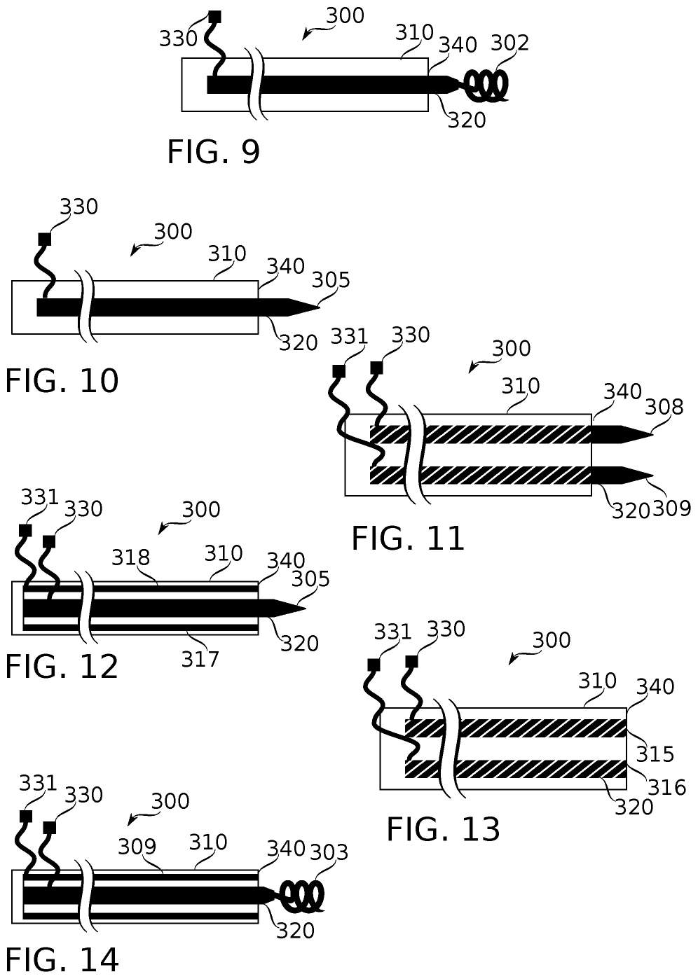

The positioning of block 110 may comprise resting one or more electrodes on tissue targeted for modification, and/or inserting one or more electrodes into tissue targeted for modification. FIGS. 9-20 illustrate different electrode probe designs, some of which insert into tissue, some of which rest upon tissue, and some of which combine the two in different electrodes and/or electrode portions.

At block 111, in some embodiments (FIG. 1B), tissue targeted for modification (e.g., on the valve annulus perimeter) is mechanically deformed. In some embodiments, this is performed by manipulating the position of one or more electrodes already inserted into the target tissue in block 110. For example, the electrodes may be squeezed together, squeezing tissue between them as well. Preferably, the electrodes are oriented (e.g., substantially tangential to the valve annulus perimeter) so that squeezing them together shortens the valve annulus perimeter. Additionally or alternatively, electrodes are rotated (individually and/or as a group), causing the tissue to distort such that distances along the perimeter of the valve annulus are shortened.

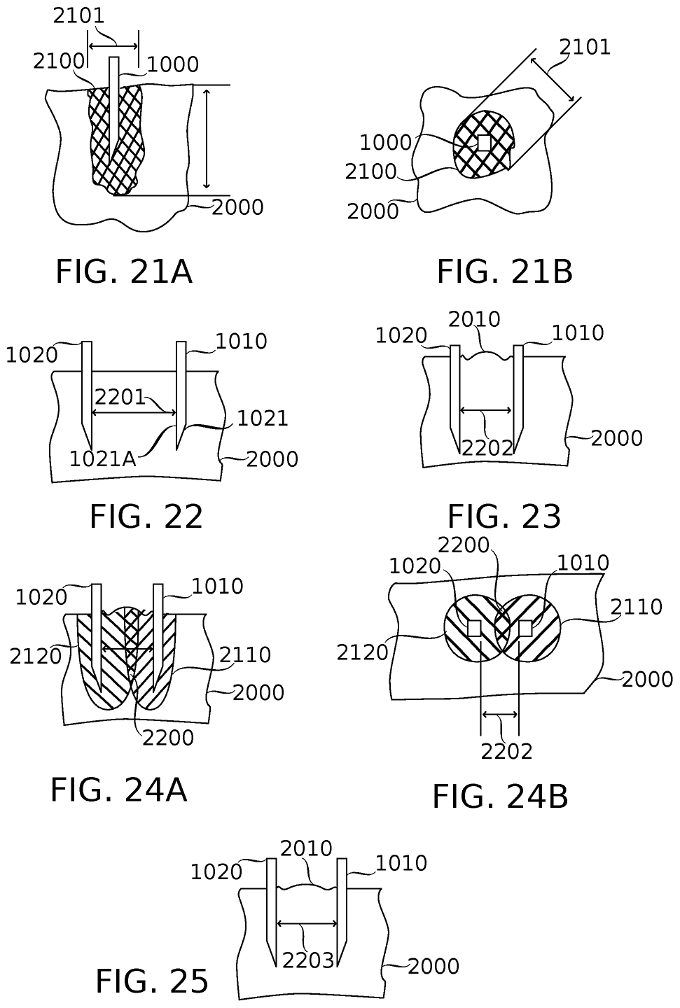

FIGS. 21A-28, 31A-32 and 35A-37C illustrate probes operable to deform target tissue by squeezing it between two electrodes. FIGS. 33-34D illustrate a probe comprising an electrode which can be rotated to deform target tissue.

At block 112, in some embodiments, tissue is subjected to structural disruption; for example ablation by the application of radio frequency (RF) energy, or another structurally disruptive energy provided by the probe. Optionally, the operations of blocks 112 and 111 occur at least in part simultaneously. In some embodiments, as energy delivery proceeds, tissue may become partially “plasticized”, allowing more movement by mechanical deformation. In some embodiments, heating induced by the delivery of structurally disruptive energy is also used to drive the induced mechanical deformation, e.g., by using a probe comprising a shape-memory alloy which activates to move heating electrodes when it is itself heated above its transition temperature.

Using the same member to both mechanically deform and structurally disrupt tissue has potential advantages for device simplicity of construction and/or operation. In this case, at least one of the electrodes also acts as a piercing element, which may in turn be a jaw which acts to stretch and/or compress tissue, and/or which is rotated to apply torsion to tissue.

However, for the method of FIG. 1B, there is no particular limitation that mechanical distortion of tissue be performed by the same electrode(s) used to deliver structurally disruptive energy. For example, a pliers operated through a different catheter than the catheter via which structurally disruptive energy is applied may be used to gather tissue. In this case, the application of structurally disruptive energy can be performed, for example, either using a surface contact electrode, or an electrode that penetrates into (pierces) the tissue itself.

In some embodiments, jaws of a pliers that distorts tissue are inserted to tissue in positions outside the targeted zone of structural disruption, squeezed to distort tissue including the target zone itself, and then energy applied to structurally disrupt tissue within the targeted zone. This has the potential advantage of focusing mechanical forces on tissue which is left healthy, rather than potentially weakened by the application of structurally disruptive energy.

Reference is now made to FIG. 2A, which schematically illustrates an electrical monopolar ablation system 90. Reference is also made to FIG. 2B, which schematically illustrates a bipolar ablation system 91. Ablation systems of this general type are known for use in ablating cardiac tissue for treatment of heart conditions such as atrial fibrillation.

The method of FIG. 1 is optionally carried out using an RF ablation electrode such as is known in the art, e.g., an RF ablation electrode configured generally as described in relation to FIGS. 2A-2B.

In some embodiments, ablation system 90, 91 comprises RF generator 71, configured to generate radio frequency (RF) energy used to perform ablation, and to define parameters of the RF energy, for example its voltage and/or current; and/or the amplitude, frequency and/or pulse shapes of the delivered RF energy.

RF generator 71 is electrically connected with (wired to) ablating catheter 100. Ablating catheter 100 comprises ablating electrode 101 through which ablating RF energy is delivered to targeted tissue via a conductor 74, with ground return being via electrical interconnection with electrically conducting (e.g., metallic) ground electrode 72 (FIG. 2A), or secondary catheter electrode 101B (and conductor 75; FIG. 2B). Ground electrode 72 may comprise, for example, a plate positioned below a reclining patient during the medical procedure; or one or more electrically conductive pads attached, for example, around the patient's arm/hand.

The RF systems of FIGS. 2A-2B are described as examples of equipment which may be used to perform valve annuloplasty. Ablation can be performed using systems which induce tissue scarring via another, for example, cryoablation, or thermal ablation using focused ultrasound. Optionally, the RF systems of FIGS. 2A-2B are run in a sub-ablative mode which disrupts tissue structure without necessarily inducing cellular death.

Reference is now made to FIGS. 3-5, which schematically illustrate distal elements of a steerable catheter 301 used to administer structurally disruptive energy (optionally an example of an RF ablating catheter 100, or an ablating catheter using another ablation energy type; for example a cryoablation catheter or a focused ultrasound ablation catheter), according to some embodiments of the present disclosure.

In some embodiments of systems used to perform the method of FIG. 1, catheter steering is provided to assist guiding an energy delivery probe into contact with parts of the valve annulus targeted for structural modification. It may be appreciated that the steering angles needed can be sharp, given the relatively confined space, especially compared to the large extent of the target area. Moreover, steering angle preferably is selectable so that the catheter approaches targeted surface areas at a perpendicular or nearly perpendicular angle. This can make it easier to establish reliable contact surface and/or pressure allowing transfer of RF energy, while also reducing the tendency of the probe to “slip” along the targeted surface when the two meet at more oblique angles. Furthermore, it is a potential advantage for there to be a stiffness to the steering system steering that results in strong, reliable contact with targeted tissue.

FIGS. 3-5 indicate elements of how a steering system with such properties may optionally be provided, in some embodiments of the present disclosure.

FIG. 3 illustrates a distal portion of an outer tube 200, made, e.g., from an electrical isolating material (e.g. PTFE, Pbax, or another electrically insulating material). Its distal tip 201 is pre-shaped to assume, when unconstrained, a relatively sharp bend, e.g., a bend of at least 70°, 145°, or 180°. Optionally, distal tip 201 comprises a spring element such as a nitinol strip/element (fully covered by inside outer tube's 200 wall) to increase its elasticity and/or mechanical rigidity properties.

Additionally or alternatively, outer tube 200 includes an articulation mechanism; for example, an embedded pulling wire which slides through a lumen inside the wall of outer tube 200 and is rigidly connected to its tip, hence enabling to control the articulation angle.

FIG. 4 illustrates inner component 300. In the example of FIG. 4, inner component 300 is used for RF delivery of structurally disruptive energy. The illustrated example of inner component 300 comprises a metal/alloy conductive component 320 (e.g. made of stainless steel, Nitinol, or another metal). Conductive component 320 is covered by an isolation layer 310 (e.g. made of PTFE, PEEK, polypropylenes, polyamide, polyimide, Pbax, or another electrically insulating material), leaving exposed at least a distally exposed region 320A, which acts as the transmitting source of RF energy. Electrical interconnection (e.g., with an RF generator 71) is made via connector 330. Inner component 300 optionally is structured as appropriate for another energy type; for example, it may be a cryoablation catheter or a focused ultrasound ablation catheter. Other designs of inner component 300 for use in RF disruption of tissue are described, for example, in relation to FIGS. 9-20.

FIG. 5 illustrates catheter 301 having inner component 300 slidably positioned within outer tube 200. Once inner component 300 is connected (using connector 330) to the RF generator, its distal tip can be activated and consequently disrupt the target location. Other inner component types may be connected to different sources of disruptive energy and/or material; for example cryofluid in the case of a cryoablation catheter, or an ultrasound transducer controller in the case of a focused ultrasound ablation catheter.

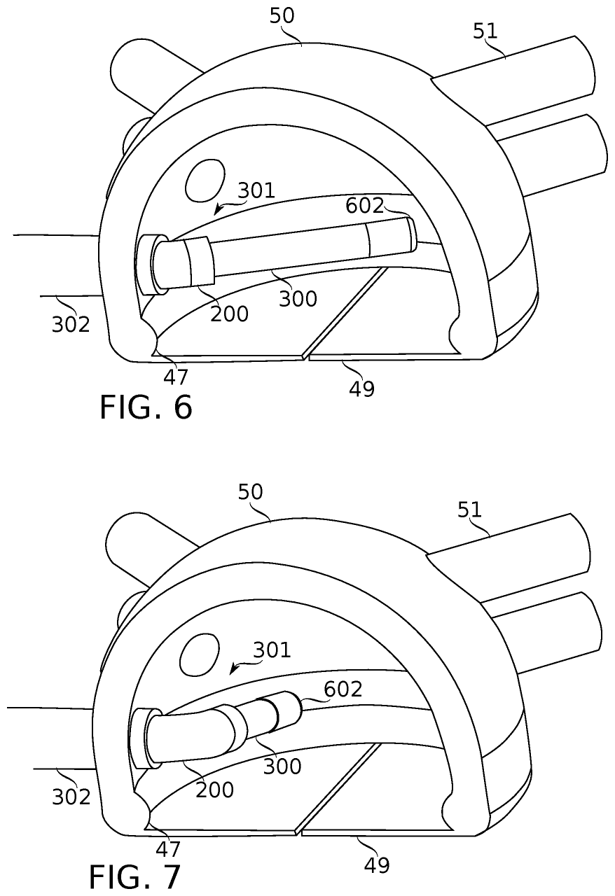

The material of distal tip 201 is sufficiently stiff that it can deflect inner component 100, but sufficiently elastic and flexible that can itself be reversibly straightened, e.g., upon sliding withdrawal into a guiding catheter 302 (shown in FIGS. 6-7, for example). As distal tip 201 is released from confinement (e.g., by advance out of a guiding catheter 302), it re-assumes its pre-shaped bend. In some embodiments, a diameter of curvature of distal tip 201 is between 10-35 mm. In some embodiments, the curvature begins about 3-15 mm proximal to the tip.

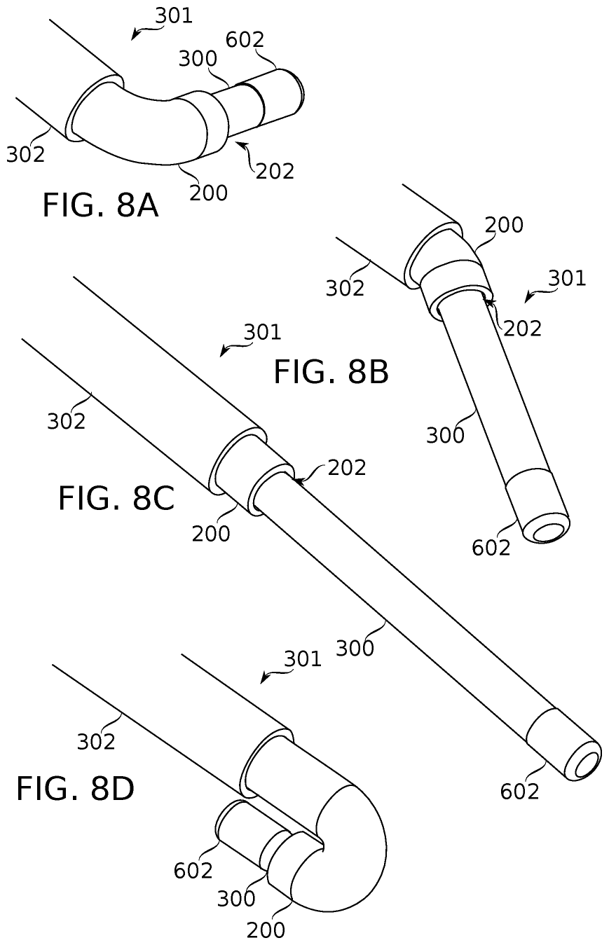

Reference is now made to FIGS. 6-7, which schematically illustrate steering of catheter 301 within a heart chamber (left atrium 50), according to some embodiments of the present disclosure. Further reference is made to FIGS. 8A-8D, which schematically illustrate additional configurations of catheter 301, out of the variety which enables delivery structurally disruptive energy to any location along the annulus.

FIG. 6 shows catheter 301 inserted to a left atrium through guiding catheter 302 (positioned in the septal wall. By linear and rotational movements of outer tube 200, and linear movements of inner components 300, the physician is enabled to place the energy delivering segment 602 (which may be for example, a conductive component 320) against the target tissue (e.g. the annulus or the atrial/myocardial wall) and activate it to disrupt tissue. In some embodiments (e.g., as described in relation to FIGS. 9,14, and/or 18), the physician may also rotate/torque inner component 200, when it has a helical or a drill-like tip shape which enables intra tissue disruption).

Afterwards the physician can direct the energy delivering segment to another location over the annulus (as shown in FIG. 7) and disrupt its structure.

By controlling/positioning outer tube 200 and inner components 300, the physician can reach and disrupt any location along the annulus. FIGS. 8A-8C show configurations of catheters in other states of unsheathing:
Fully unsheathed (FIG. 8A), allowing outer tube 200 to assume, e.g., a full 90° turn. Optionally, another fully unsheathed angle is configured; for example, the example of FIG. 8D shows an outer tube 200 which bends through a full 180° when unsheathed from guiding catheter 302. By the bending of outer tube 200, distal aperture 202 of outer tube 200 also re-oriented to a new angle relative to a longitudinal axis of the distal portion of guiding catheter 302.
Partially unsheathed (FIG. 8B), allowing partial (e.g., oblique, though partial deflection might be a 90° deflection if the maximum bending angle is greater than 90°) deflection of outer tube 200 away from the longitudinal axis of the distal end of guiding catheter 302.
Fully sheathed or almost fully sheathed (FIG. 8C), such that distal aperture 202 remains oriented perpendicular to the longitudinal axis of the distal end of guiding catheter 302.

Reference is now made to FIGS. 9-20, which schematically illustrate alternative designs of inner component 300, for use with an RF ablating system, according to some embodiments of the present disclosure. In some embodiments, an inner component 300 design as described in relation to FIGS. 9-20 is provided as alternative implementation of an energy delivering electrode 100 in the system of FIGS. 2A-2B.

In overview, each of the examples of FIG. 9-10 illustrate at least an inner component 300 comprising one or more conductive components 320, attachable to RF power by respective connectors 330, 331. Over most of their length, conductive components 320 are covered by electrical insulation 310; optionally comprising a polymer tube of one or more lumens, and/or an electrically insulating coating. The designs shown differ from one another in details such as how conductive components 320 are terminated at their tips (which are in each case electrodes from which RF energy is delivered), how many conductive components 320 are provided, and/or how they are arranged. In addition to the specific combinations shown, features illustrated can be combined from among different embodiments insofar as they are mutually compatible; e.g., the different tip types (helical-tipped, conical-tipped, screw-tipped, blunt-tipped, and rounded tipped, for example) can be provided in any combination to conductive components 320 (monopolar, bipolar coaxial, and bipolar non-coaxial, for example).

In FIG. 9, inner component 300 comprises helical tip 302, attachable to RF power by monopolar connection 330 via conductive component 320.

Rotating inner component 300 while advancing it screws helical tip 302 into the target tissue (e.g. into the connective tissue of the annulus). This may be performed as a preliminary to assure stable contact and/or deeper scar penetration before disruption is performed by supplying RF energy through helical tip 302. Optionally the helical distal tip 302 is sharp to ease penetration into (piercing) the target location (e.g. by engaging the tissue and then rotating the inner component 300 relative to outer tube 200).

Optionally, inner component 300 includes a distal surface 340 which restricts penetration depth of energy delivery tips (e.g., tip 302 of FIG. 9) into the target tissue. Distal surface 340 is also indicated in each of FIGS. 10-15 and 17-20, where it performs the same depth limiting function, at least for electrode tips that are intended to penetrate tissue to some depth.

FIG. 10 schematically illustrates a monopolar inner component 300, having a distal tip 305 which is sharp and can penetrate into the tissue linearly.

FIG. 11 schematically illustrates a bipolar inner component 300 tipped with two electrodes 308, 309.

FIG. 12 schematically illustrates a bipolar inner component 300 having a tubular electrode 317 (which does not penetrate into the tissue) and electrode 318 (which have a sharp tip and penetrates into the tissue). Tubular electrode 317 may comprise a solid wall, a helical construction, and/or conductive mesh construction.

FIG. 13 schematically illustrates a bipolar inner component 300 having electrodes 315 and 316 which engage the target tissue by end contacts of conductive component 320, without penetrating tissue.

FIG. 14 schematically illustrates a bipolar inner component 300 having a tubular electrode 304 which doesn't penetrate into the target tissue, and electrode 303 which has a distal helical tip having a sharpened end that penetrates into the target tissue. Tubular electrode 304 may comprise a solid wall, a helical construction, and/or conductive mesh construction.

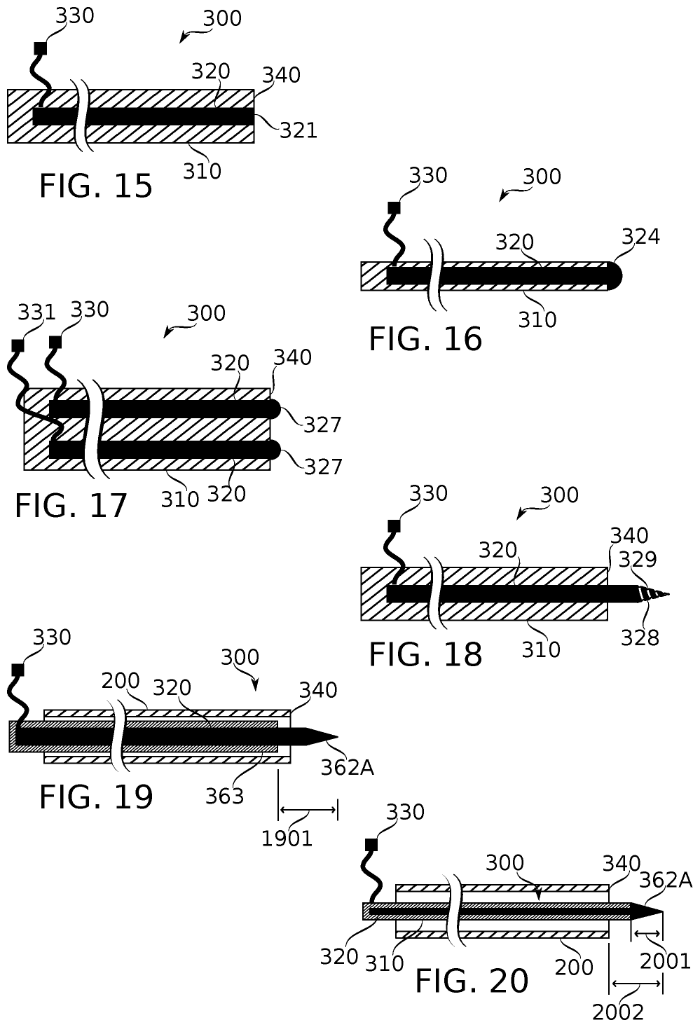

FIG. 15 schematically illustrates a monopolar inner component 300 having a single conductive component 320 terminating in an electrode contact surface 321 flush with surface 340.

FIG. 16 schematically illustrates a monopolar inner component 300 having a single conductive component 320, which is distally ended with an atraumatic round segment 324.

FIG. 17 schematically illustrates a bipolar inner component 300 having a pair of inner components 320 which are both distally ended with atraumatic round segments 327.

FIG. 18 schematically illustrates a monopolar inner component 320 having an electrode with a distal screw-threaded sharp tip 328. Helical groove 329 potentially eases the pre-activation penetration into the target tissue by rotation and drilling.

FIG. 19 schematically illustrates an inner component 300 together with an outer tube 200, allowing variably selectable penetration depth of electrode tip 362A. Conductive member 320 can be slid longitudinally relative to distal surface 340 of insulating tube 363 (insulating tube 363 is an instance of an insulation layer 310), and optionally locked into place, e.g., by locking of a control element on a proximal side of the device. The sliding enables defining different penetration depths by adjusting distance 1901 between distal surface 340 and the distal-most tip of the electrode 362A.

A potential advantage of this is to allow changing desired disruption depth per location selected for energy delivery; for example according to the depth of valve annulus connective tissue, the angle of approach to the tissue, and/or according to the proximity of structures which should not be disrupted such as nearby coronary arteries.

FIG. 20 schematically illustrates an inner component 300, also with selectable depth, but in this case the depth of penetration (distance 2002) is selected by adjusting the relative longitudinal position of the distal-most tip of electrode 362A relative to a distal surface 340 of outer tube 200.

This configuration allows penetration of tissue beyond the uninsulated depth 2001 of tip 362A, while retaining control of penetration depth. Layers of tissue above the uninsulated depth 2001 of tip 362A will potentially be relatively spared from damage during delivery of structurally disruptive energy.

Optionally, any of the inner components 300 (e.g., of FIGS. 9-20) includes one or more lumens operable as working channels; for example, useful for translation of guide wire/s, injection of irrigation fluid, suction, and/or injection of radiopaque liquid. A thermocouple sensor is optionally provided to any of the inner components 300 at or near their tip, and is wired distally along inner component to a connection with an outer controller. This allows monitoring with the potential advantage of allowing the physician to stop delivery of energy once the tissue is heated to a certain threshold temperature. Optionally (using a controller), the disruption can be stopped automatically once the tissue reached a certain threshold temperature.

Additionally or alternatively, tissue impedance is measured to track progress of disruption, optionally via one or more of the electrode or electrodes used for delivery of energy. For example, a change in tissue impedance (an increase, generally; for example an increase of about 7-fold) as it is ablated change can be used to determine when ablation has completed, or reached a target partial intermediate state of tissue disruption.

The change is optionally used to automatically halt disruption of tissue after a certain impedance threshold is reached (e.g., a magnitude above about 2200Ω), and/or a certain magnitude of impedance change has occurred (for example, an increase in impedance magnitude of about 1800Ω-1900Ω, or an increase by a factor of 5, 6, 7, 8, or another factor). The impedance may be measured at an RF frequency; for example, a frequency of around 500 kHz. Additionally or alternatively, impedance may be used to confirm initial penetrations of electrodes into tissue (e.g., a decrease in impedance, for example, a decrease to about 400Ω), and/or to confirm that the distance between electrodes has been closed to a smaller distance (e.g., a reduction in impedance magnitude, for example a decrease by about 100Ω and/or to a value of about 300Ω). Using the electrode(s) which deliver structurally disruptive energy also for measurement has the potential advantage of eliminating a need for providing additional signal wiring to the device.

The values for impedance changes just given have been observed in tissue using electrodes with about 0.4×0.4 mm cross sections, about 4 mm penetration depth, and using an RF frequency of about 500 kHz. Different values may be observed using different electrodes and/or operating conditions.

### Tissue Remodeling by Structurally Disrupting Mechanically Deformed Tissue

Reference is now made to FIGS. 21A-21B, 22, 23, 24A-24B, and 25 which schematically illustrate a method of shaping tissue by delivering structurally disruptive energy while the tissue is mechanically deformed.

Cross-sectional FIG. 2/A and top view FIG. 21B, illustrate a zone of scarring 2100 resulting from the activation (e.g., by transmission of RF energy) of device 1000 while placed within tissue 2000. The zone of disruption may be approximately characterized by a depth of disruption 2000, and a diameter of disruption 2101. Although treated as cylindrical for purposes of description, it should be understood that the zone of disruption is not necessarily cylindrical—the tissue and the heat transfer is not necessarily homogeneous. The scenario illustrated is similar to one created by any of the embodiments of an inner component 300 which comprises a tissue-penetrating electrode. Following disruption, the zone of disruption 2100 is prone to shrinkage (e.g., potentially as a result of fluid loss, cell death, and/or coagulation).

From results of their in vivo and ex-vivo experiments, the inventors have recognized a further effect which can be utilized both to fixate and/or to shrink (or shape) tissue such as heart valve annular tissue. Tissue which is originally elastic (pre-disruption) can be forced to undergo a certain amount of plastic deformation (additional to that which its own shrinkage would normally induce) by placing it under mechanical deformation while administering disruption energy (and more particularly, disruption energy which produces coagulation). The use of compression in particular provides a potential advantage for the application of annuloplasty, e.g., by allowing sufficient shrinkage of the valve circumference using a smaller number of applications, and/or a smaller region of tissue disruption.

FIGS. 22-24B illustrate one method of generating plastic deformation by compressing tissue during lesioning.

In FIG. 22, in some embodiments, electrode 1010 and electrode 1020 are inserted into tissue 2000, with a distance 2201 between them.

In FIG. 23, the distance between the electrodes is reduced to a shorter distance 2202, while the electrodes remain in the tissue 2000. This produces mechanical compression of tissue between the electrodes 1010, 1020.

Lesioning is then performed. FIGS. 24A, 24B illustrate the respective zones of disruption 2110 and 2120 for electrodes 1010, 1020 upon their operation (e.g., transmission of RF energy) to disrupt nearby tissue. Distances and energy delivery parameters are optionally selected so that zones 2110, 2120 share a common section 2200, resulting in a continuous zone of disruption.

Lesioning can have effects which break down some portion of tissue elasticity (e.g., by lysis of cells), while other effects may tend to tighten and stiffen cellular and/or extracellular components (e.g., by denaturing proteins) so that they resist returning to their original shape. In effect, the lesioning fixes the tissue into a new preferred configuration, more similar to the compressed shape used during lesioning than the shape the tissue previously was predisposed to assume.

Upon release of external forces (e.g., relaxation of compression between electrodes 1010 and 1020), the tissue potentially assumes a new equilibrium state, e.g., one in which distance 2203 between electrodes 1010, 1020 (FIG. 25) is now shorter than distance 2201. Insofar as some elastic memory of the original tissue shape remains, distance 2203 will typically also be larger than distance 2202. Removing the electrodes leaves behind the disrupted and compression-remodeled tissue.

If electrodes over-compress tissue between them (or over-stretch tissue), there is a risk that structural disruption of tissue will result in cutting damage which may potentially negate the intended outcome.

Alternatively, should the level of applied energy (e.g., RF energy) be too low, the zone of disruption may not extend into some of the most deformed regions (e.g., the zone of disruption may not be continuous between the electrodes). This can result in a reduction in the magnitude of the targeted effect. However, it is important to select an energy level which is safe and does not induce an electrical disorder (e.g. ventricular fibrillation) in the patient. In general, choosing lower power for a longer duration is safer (E.g. 10 watts for 12 seconds is safer than a same energy level alternative of delivering 30 watts for 4 seconds).

A potential set of effective and parameters for tissue disruption comprise inserting into tissue electrodes with an initial distance 2201 of about 4 mm, compressing tissue between them until their distance is about 1.7 mm, and disrupting tissue structure with RF energy at power of 7 watts for 16 seconds. These parameters were derived by experiment using electrodes having a width of 0.4 mm, and penetrating around 4 mm into the tissue.

It may be noted that each of electrodes 1010, 1020 are illustrated with a beveled tip 1021 in FIGS. 22-25. This gives the electrode cutting surfaces a sidedness. Orienting the bevel with the uncut (or less-cut) side 1021A inward potentially reduces risk of cutting compressed tissue.

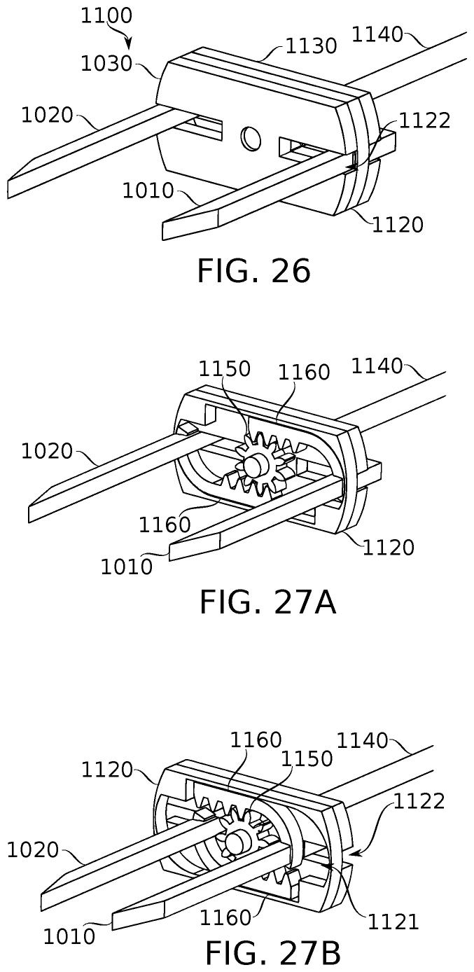

Reference is now made to FIGS. 26 and 27A-27B, which schematically illustrate electrode pliers 1100, according to some embodiments of the present disclosure.

Pincer 1100 can be provided at the distal tip of a flexible low-profile catheter for use in disruption of tissue structure via an endovascular approach.

Pincer 1100 (FIG. 26), in some embodiments, comprises electrodes 1010, 1020; which in the example shown also act as the tissue grasping (gripping and squeezing) elements of pliers 1100. Moreover, electrodes 1010, 1020 are shaped to penetrate the tissue to be grasped. Optionally, some portion along the lengths of electrodes 1010 1020 is insulated, e.g., to allow selective disruption of tissue at certain depths, for example as described in relation to FIG. 20.

Normally, covers 1130 are rigidly connected with frame 1120. FIGS. 27A-27B show one cover 1130 removed from pliers 1100 to allow illustrating inner workings of pliers 1100.

In some embodiments, a rack-and-pinion arrangement (FIGS. 27A-27B) is used to actuate the electrodes. In the example shown, each electrode 1010, 1020 is separately provided with a rack (linear gear) 1160 to which it is rigidly attached. Pinion 1150 is optionally a spur gear which rotates to move the racks and translate the electrodes toward each other, or away from each other. Frame 1120 and covers 1130 are shaped to hold and guide these components, e.g., to hold them within recess 1121, and to guide movement of the electrodes along slots 1122.

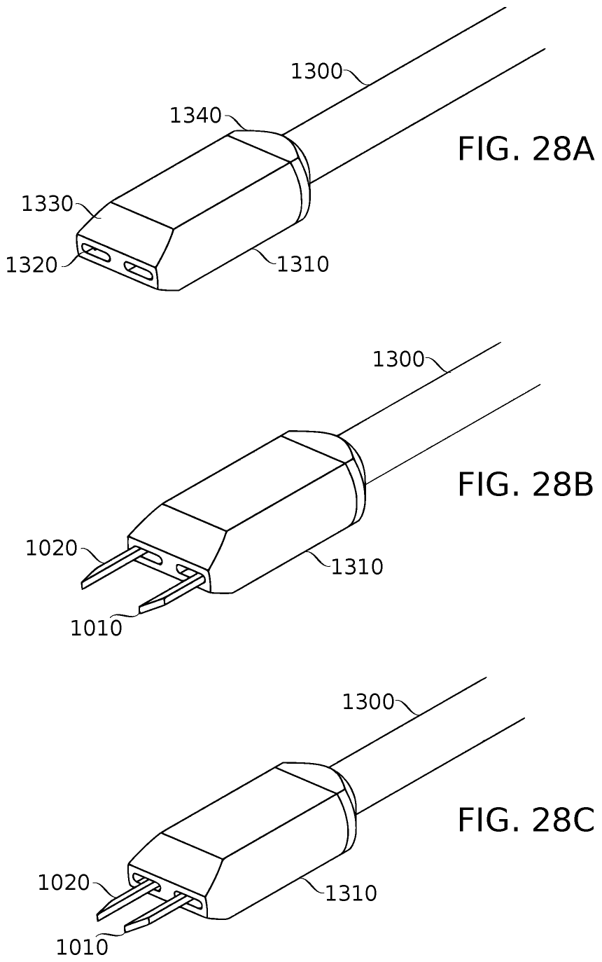

Spur 1160 is rigidly connected with an elongated member 1140 (e.g., a rod or wire). Once the electrodes 1110, 1120 of the pliers are embedded in tissue, rotating elongated member 1140 turns pinion 1150, since the rest of the body of pliers 1100 is anchored. Additionally or alternatively, as described in relation to FIGS. 28A-28C, elongated member 1140 may itself be encased within a tube 300, allowing electrode distance adjustment by rotation of elongated member 1140 relative to a device casing even when there is no external resistance to rotation.

In FIG. 27A, electrodes 1020, 1010 are positioned in their widest spacing; in FIG. 27B, rotation of pinion 1150 has brought them closer together by meshing with the linear gears 1160. Whether the device results in stretching or compression of tissue depends on how it is used. Penetration in the wide-spaced configuration of FIG. 27A followed by reduction of inter-electrode distance (FIG. 27B) will tend to compress tissue. Penetration in the narrow-spaced configuration of FIG. 27B followed by increase of inter-electrode distance (FIG. 27A) will tend to stretch tissue.

In some embodiments, the components of pliers 1100 are metal; produced, for example by laser cutting of flat stock to produce plates, gearing, and/or electrodes. In some embodiments, electrical isolation from the environment is provided by a tip casing 1310, e.g., as described in relation to FIGS. 28A-28C. Optionally, electrical isolation of one or more covers 1130, frame 1120, and/or elongated member 1140 from the environment and/or from components conveying RF energy is provided by coating surfaces of these elements with an electrically insulating polymer (e.g. Perylene-c or PTFE).

Optionally, frame and covers 1130 are made entirely of an insulating material (e.g., polymer). Where pinion 1150 is itself a conductive part, it may be used to transmit RF power to the electrodes 1010, 1020 via electrical conduction through their respective (electrically conductive) linear gears. Alternative, electrodes 1010, 1020 may be directly connected to power leads. In this case, pinion 1150 is optionally itself formed from polymer, allowing electrodes 1010, 1020 to operate in a bipolar mode (when each is provided with its own power lead), instead of as two different portions of a monopolar electrode.

The combination of pliers functionality and electrode functionality within electrodes 1010, 1020 has a potential advantage in simplifying the device design. Device operation may also be simplified. However, it should be understood that these functions are optionally performed by separate components.

For example, grasping is optionally performed by pliers which are first operated to grip and reshape (e.g., compress) tissue (with or without initial penetration of tissue). Once the tissue is reshaped, electrodes may be placed upon or inserted into tissue and operated to disrupt it. This has a potential advantage by optionally decoupling the region of greatest lesioning energy from the area of greatest mechanical stress, potentially reducing likelihood of tearing due from tissue weakening during or after structural disruption of tissue.

Reference is now made to FIGS. 28A-28C, which schematically illustrate a covered catheter tip casing encasing the mechanism of FIGS. 27A-27C, according to some embodiments of the present disclosure. This may be used to sheath electrodes 1010, 1020, e.g., to sheath during navigation of the catheter to the target tissue, to avoid injury due to sharp tip of the electrodes. Once there, electrodes 1010, 1020 can be exposed and distance adjusted as needed.

In some embodiments, tip casing 1310 comprises polymer (e.g. PEEK, PTFE, etc.) and/or metal (e.g. stainless steel, titanium, and/or another biocompatible metal) having a polymeric coating (e.g. Perylene-c or PTFE).

As a result, casing 1310 is electrically isolating (hence during the administration of structurally disruptive energy, there is insignificant electrical leakage to the non-target tissues). Moreover, in embodiments where tip 1310 is polymer made (and not radio-opaque), electrodes 1010, 1020 (which are metallic and relatively radio-opaque) are well observed under fluoroscopy. Optionally, tip casing 1310 includes a radiopaque marker, allowing observation under fluoroscopy of the positioning of electrodes 1010, 1020 relative to casing 130 (e.g., inside or outside casing 1310).

To assist in visualization via ultrasound, casing 1310 may be provided with a surface texture such as grooving, which may increase its echogenic properties. Optionally or additionally, slow flushing of fluid (e.g., saline) through casing 1310 is used to increase echogenesis to assist in localizing the position of casing 1310.

In a retracted configuration, (e.g., used for navigation), tip 1310 covers the electrodes (FIG. 28A). Once the distal end of casing 1310 is positioned against the target tissue, electrodes 1010 are unsheathed, e.g., by advancing out of casing 1310 and/or pulling casing 1310 backwards. For example, tube 1300 may be rigidly connected with tip 1310, so that withdrawing it exposes electrodes 1010, 1020 (FIG. 28B).

Once electrodes 1010, 1020 are positioned inside the target tissue, the operator can decrease distance between them, as shown in FIG. 28C (e.g., using the inner driving mechanism 1100 described in relation to FIGS. 27A-27C). The device is activated, e.g., using the RF energy.

Regarding other aspects of this configuration:

In some embodiments, tube 1300 comprises a polymer material (e.g. Pbax or PTFE). Optionally, the polymer material is metal reinforced (e.g. using metal braiding or helix/es) to support the needed mechanical and electrical properties of the catheter (i.e. maneuverability/flexibility, rotatability/torque-ability, push-ability and electrical isolation).

In some embodiments, casing 1310 includes distal taper 1330 and proximal taper 1340, with the potential advantage of assisting smooth translation of the catheter smoothly forward and backwards through a guiding sheath (and/or a body lumen). Casing 1310 includes slits 1320 through which electrodes 1010, 1020 can protrude when unsheathed, and along which they can slide, according to remote actuation commands. The width of slit 1310 is matched to the width of electrodes 1010, 1020 width (e.g., to within a tolerance of about 0.1-0.2 mm around electrodes which may themselves be, for example, about 0.4 mm in width). The resulting small aperture size may allow a minimal volume of blood to penetrate into the tip. This potentially helps to reduce leakage of the electrical RF energy (such a leakage is basically a noise which complicates the ability to control the administration of structurally disruptive energy in a repeatable, durable, and/or stable manner). In some embodiments, sealing is assisted by sliding and/or elastic gaskets provided on the inner side of slits 1320, through which electrodes 1010, 1020 penetrate when unsheathed.

Casing 1310 provides another potential advantage by preventing accidental penetration of the valve leaflets or other non-targeted tissue by electrodes 1010, 1020 while casing 1301 is being moved within the body. For example, electrodes 1010, 1020 are extended directly into tissue after verifying (e.g., using transesophageal echocardiography or intracardiac echocardiography) that casing 1310 is positioned against the valve annulus tissue targeted for remodeling. After the tissue structure has been disrupted, electrodes 1010, 1020 are withdrawn back into casing 1310 before it is moved again (e.g., moved to a new treatment position or withdrawn from the body). Optionally the distance between electrodes 1010, 1020 is reset to a wide position after withdrawal from tissue, and while they are retracted into casing 1310.

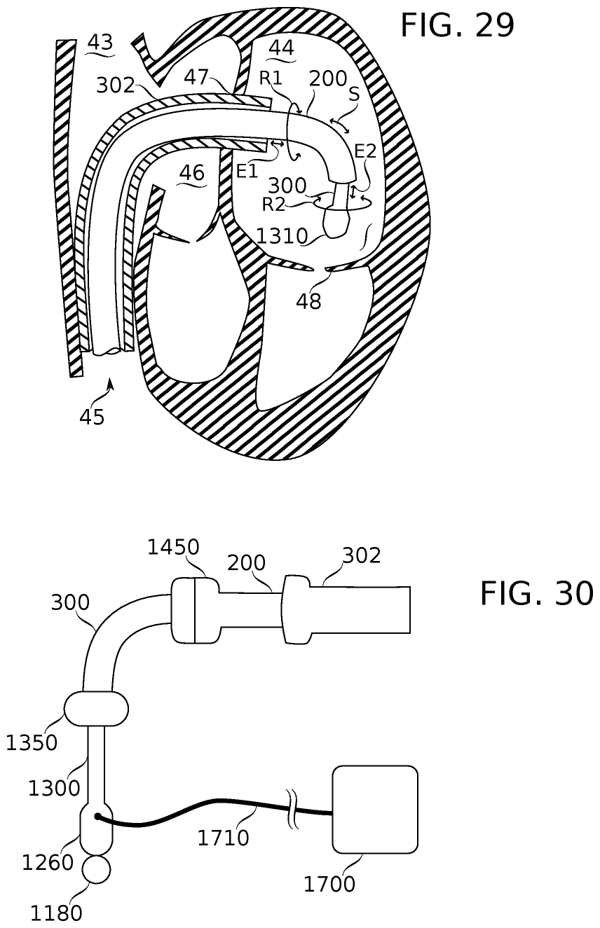

Reference is now made to FIG. 29, which schematically illustrates positioning via an endovascular approach of the distal portion of a catheter used for annuloplasty of a mitral valve 48, according to some embodiments of the present disclosure.

In some embodiments, transseptal guiding catheter 302 is guided to the right atrium 46 via the inferior vena cava 45. Guiding catheter 302 is guided to penetrate the interatrial septum 46 (preferably via the fossa ovalis) to gain minimally invasive access to the left atrium 44. The catheter comprising outer tube 200 and inner component 300 (comprising energy delivery element 1310) is inserted through guiding catheter 302 into the left atrium 44. Alternatively, in some embodiments, access to the heart is via the superior vena cava 43. This is potentially advantageous for treatment targeting the valve annulus of the tricuspid valve.

Optionally or alternatively, guiding catheter 302 itself comprises a distal steering section (instead of curved pre-shaped form) and/or sleeve 1400 has a curved pre-shaped distal segment (instead of the steering segment being described above).

Controllable degrees of freedom of outer tube 300 include rotation R1 relative to guiding catheter 302, and longitudinal advance/retraction E1 relative to guiding sheath 1500.

In some embodiments, outer tube 300 also comprises a steering segment S which bends through a range of angulations, for example by active steering (e.g., controlled by shortening a control wire), and/or via unsheathing of a pre-shaped curved form, for example as described in relation to FIGS. 3-8D. In some embodiments, steering is performed by a steering mechanism provided to guiding catheter 302 itself.

Controllable degrees of freedom of inner component 300 include linear translation E2, relative to sleeve 302, and rotation R1 relative to sleeve outer tube 200. In combination, the controllable degrees of freedom allow placing catheter's tip 1310 at selected positions, orientations and angulations along the annulus, from which electrodes may be deployed to penetrate the annulus and perform structural disruption of tissue.

FIG. 30 demonstrates an optional proximal side of the catheter (which is placed outside the patient's body). Outer tube 300 is inserted into guiding catheter 302 and is provided with a handle 1450 used for control of longitudinal advance relative to guiding catheter 302 and steering (angulation) of its distal segment.

Inner component 300 in turn is positioned inside outer tube 200. Handle 1350 controls longitudinal advance and rotation of inner component 300 relative to outer tube 200.

In some embodiments, handle 1260 drives deployment of electrodes 1020, 1010 by longitudinal movement relative to tip casing 1310, e.g., via connection with tube 1250, which in turn connects distally to tube 1300. Rotation of handle 1180 rotates elongated member 1140, to drive electrodes 1010, 1020 laterally, for example as described in relation to FIGS. 26-27B.

Additionally, handle 1260 (or handle 1180) contains a hole through which electrical cable 1710 passes, allowing connection of electrodes 1010, 1020 RF generator 1700.

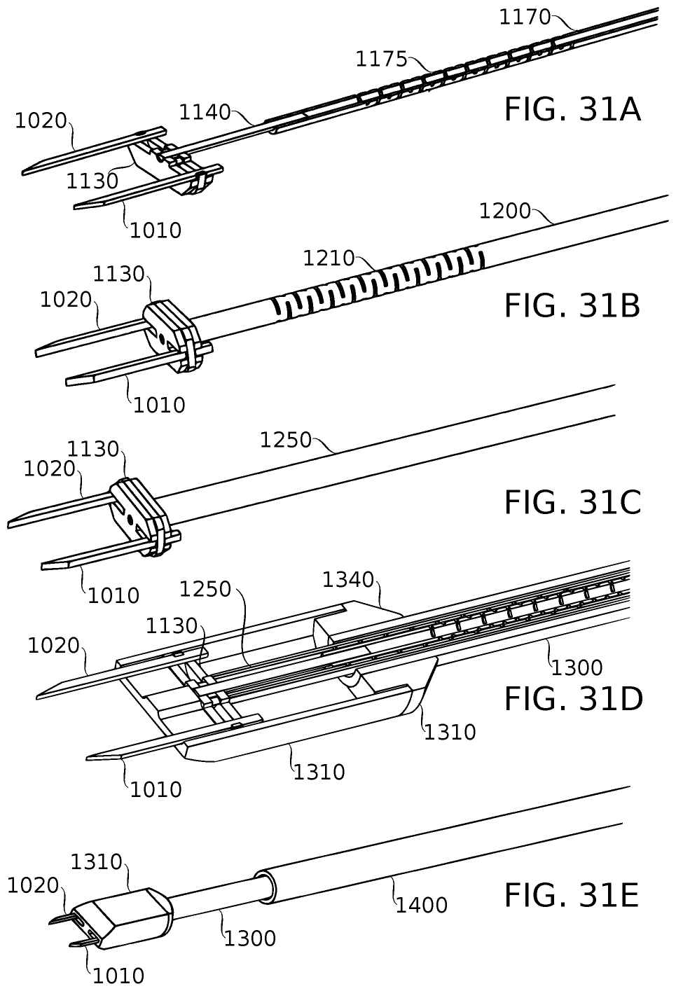

Reference is now made to FIGS. 31A-31E, which schematically illustrate different constructed layers of an adjustable-width catheter, according to some embodiments of the present disclosure. In the cross-section drawn in FIG. 31A, wire 1140 is shown rigidly connected on its proximal side to a distal side of tube/shaft 1170. The rigid connection is made, for example, by crimping tube 1170 over wire 1140, by gluing them, and/or by welding them using laser cutting technology.

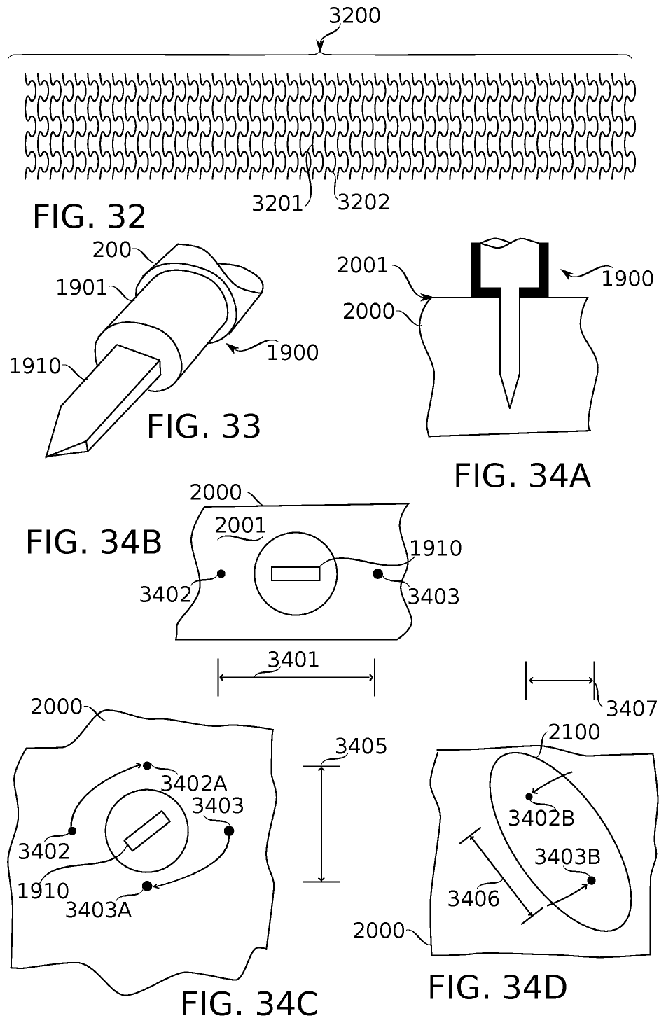

In some embodiments, tube 1170 is constructed of metal (e.g. stainless steel or nitinol), and provided with a flexible segment 1175. Segment 1175 may be conferred flexibility, for example, by making laser cutouts (slits, for example, or an interlocking pattern for example as described in relation to FIG. 32) in sold-walled tubing. Alternatively, segment 1175 may be conferred flexibility by construction from metal braiding and/or one or more wire helices. In some embodiments, the metal braiding and/or wire helices are reinforce a tube of polymer construction (e.g. PEEK, polyimide, PTFE, and/or Pbax).

FIG. 31B illustrates the next-outer layer. Tube 1200 is assembled over tube (or shaft) 1170, and rigidly connected with cover 1130 (e.g. using laser welding). Rotating tube/shaft 1170 relatively to tube 1200 drives electrodes 1010, 1020 in lateral directions to bring them closer or drive them apart. Tube 1200 contains a flexible segment 1210; produced, for example, as described for flexible segment 1175.

At least one of tube or shaft 1170, 1200 is metallic, allowing conduction of electrical energy along the catheter to electrodes 1010 & 1020 (e.g., via proximal cover 1130).

FIG. 31C illustrates insulating sleeve 1250, assembled over and rigidly connected to tube 1200. Sleeve 1250 is polymer made (e.g. PTFE or Polyolefin made) to electrically isolate the conducting internal tube. In some embodiments, sleeve 1250 is a heat shrink tube, with a potential advantage for simplifying the assemble process.

Tube 1300 (FIG. 31D) is assembled over sleeve 1250 and rigidly connected (e.g. using a glue) with tip casing 1310. Tube 1300 can slide forward/backward over sleeve 1250, allowing sheathing or exposure of the electrodes 1010, 1020.

In some embodiments, outer tube 200 (FIG. 31E) is assembled in turn over tube 1300. Tube 1300 can be driven forward/backward/be rotated relatively to outer tube 200. In some embodiments, the distal tip of sleeve 200 contains a steering segment to enable the bending it to support motions and configurations described, for example, in relation to FIG. 29.

Reference is now made to FIG. 32, which illustrates an unfolded view of a self-interlocking pattern 3200 cut to provide flexibility to tube 1170 and/or tube 1200, according to some embodiments of the present disclosure. The cuts (preferably produced using a laser cutting technique) create a series of separate yet geometrically connected links. Each individual link allows slight movement, while the links in aggregate can create bends of greater angulation. Making the cuts with interlocking elements 3201, 3202 keeps the tube from falling apart. Consequently, the tube supports a high level of maneuverability (i.e. it can pass through geometries having small radius of curvatures while having good push-ability and pull-ability) while retaining a high level of torque-ability (which is needed in order to transfer the torque, which drives the electrodes laterally, along the tube/s).

### Twist-Shrinking

Reference is now made to FIG. 33, which schematically represents an electrode configuration which inserts to tissue, twists, then disrupts its structure, according to some embodiments of the present disclosure. Inner component 1900 is an example of an inner component 300 comprising a single electrode 1910, protruding from an insulating sleeve 1901, which itself fits within outer tube 200. Electrode 1910 is shaped so that when it is inserted into tissue and twisted, it drags tissue around with it. The amount of twist may be, for example, about 90°, 135°, or another distance. The twist should, however, remain below the level of force which induces tissue to slip back into an untwisted state again.

In some embodiments, this is accomplished using a perimeter shape which extends radially more distant from the central axis of the electrode in some places, compared to other places on the perimeter which are radially closer. This creates tissue surfaces which are pushed against (rather than simply slid past) when the electrode rotates, generating a twist in surrounding tissue. As simple example of such a perimeter is a rectangular cross-sectional shape. Triangular and cross-shaped cross sections provide alternative examples. Additionally or alternatively, the electrode (or electrodes) may comprise a plurality of separated shapes which insert into tissue, for example, two, three, four, or more spikes, flat blades (e.g., oriented radially from a common center) or other shapes. A larger total surface area is potentially preferable, to reduce buildup of focal stresses that elevate the risk of tissue tearing.

Reference is now made to FIGS. 34A-34D, which illustrate a twist-and-disrupt method of valve perimeter reduction, according to some embodiments of the present disclosure.

FIG. 34A shows, in cross-section, electrode 1910 inserted into a block of tissue 2000. FIG. 34B shows the same scenario from a perspective looking down on surface 2001 of tissue 2000. Distance 3401 represents the initial distance between two locations 3402, 3403 positioned along the perimeter of valve annulus tissue which is to be adjusted.

In FIG. 34C, electrode 1910 has been twisted, resulting in torsional tissue movement indicated by arrows leading between locations 3402 and 3402A, and between locations 3403 and 3403A. Incompressible tissue volume may be diverted into tissue bulges (e.g., into open areas adjacent to tissue 2000) in response to stress. This potentially shortens the overall perimeter as it is “wound up”, including drawing tissue portions at locations 3402A, 3402B somewhat closer than they were before at locations 3402, 3403.

FIG. 34D shows the situation after disruption of region 2100 and removal of electrode 1910. Tissue at locations 3402A, 3403A has partially relaxed back to positions 3402B, 3403B; but is because of plastic deformation as a result of scarring, it does not relax all the way back to its original position. Tissue shrinkage has further reduced direct distances (distance 3406 is shorter than distance 3405, for example). In the direction of the valve perimeter, locations 3402B 34093 are separated by distance 3407, which is shorter still. The difference between distance 3407 and 3401 is somewhat larger than the overall reduction of perimeter, since some of the volume of twisted tissue was diverted laterally outward by the twist, even as other tissue was being pulled laterally inward. There may nevertheless be an overall combined effect of perimeter shortening due to both tissue shrinkage and tissue plastic remodeling “set” while the tissue was held in a twisted configuration.

It should be noted that the pinching-type compression described, e.g., in relation to FIGS. 21A-25 is optionally performed together with the torsional compression described in relation to FIGS. 33-34D. This potentially increases the amount and/or range of perimeter shrinkage which can be induced from a particular site of disruptive energy delivery.

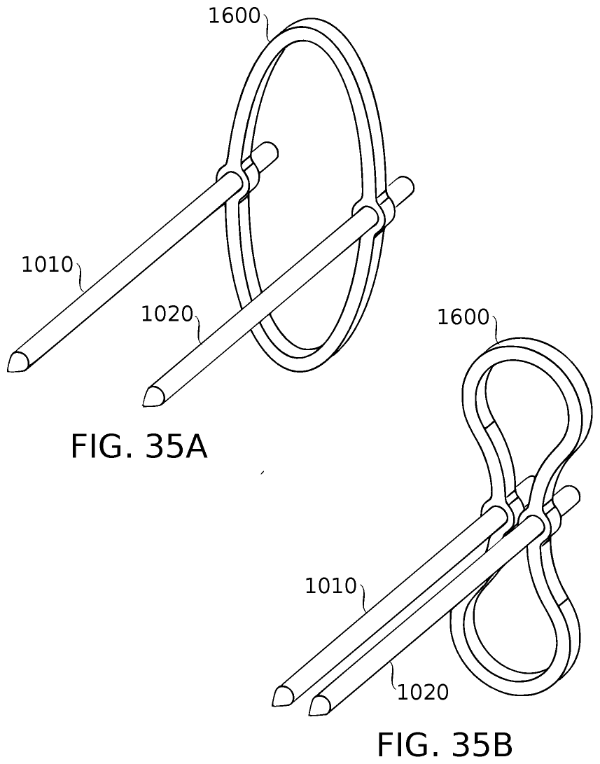

Reference is now made to FIGS. 35A-35B, which schematically illustrate configurations of an electrode assembly comprising two needle electrodes 1010, 1020 interconnected by a loop spring 1600, according to some embodiments of the present disclosure.

Electrodes 1010, 1020 are connected to respective opposite sides of loop spring 1600, which itself comprises a shape memory alloy such as nitinol. Loop spring 1600 is used as the actuator of the device. Optionally another actuator comprising a shape memory alloy is used, for example, separate leaf springs or a coiled spring.

Above the transition temperature of this alloy: when loop spring 1600 is allowed to relax, it brings electrodes 1600 close together (FIG. 35B); that is, it is “normally closed”. When loop spring 1600 is held open, electrodes 1010, 102 are separated. This element is optionally used in place of, e.g., the rack-and-pinion mechanism described in relation to FIGS. 26-27B.

In some embodiments, the shape memory allow used is set with a transition temperature above body temperature, e.g., in the range of 37°−60° C. This results in loop spring 1600 being “soft” before use. Loop spring 1600 begins cooler than the transition temperature, and bent into the open state. Due to the properties of the shape memory alloy, it will remain in that state until heated. The electrodes can be inserted to tissue in this configuration, and then operated to perform tissue structure disruption.

During operation, the electrodes rise in temperature, heating loop spring 1600 above its transition temperature. Additionally or alternatively, loop spring 1600 is self-heating by electrical resistance to current flowing through it. The heating causes it to collapse toward the closed state, drawing the electrodes toward each other. This is another way of applying external forces to influence plastic deformation effects of tissue, e.g., as described in relation to FIGS. 21A-25.

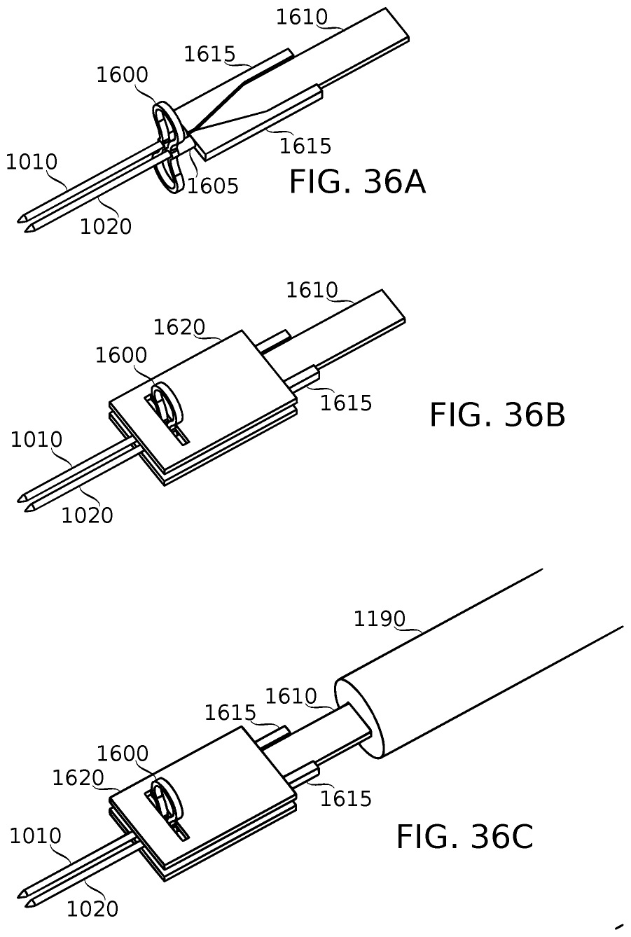

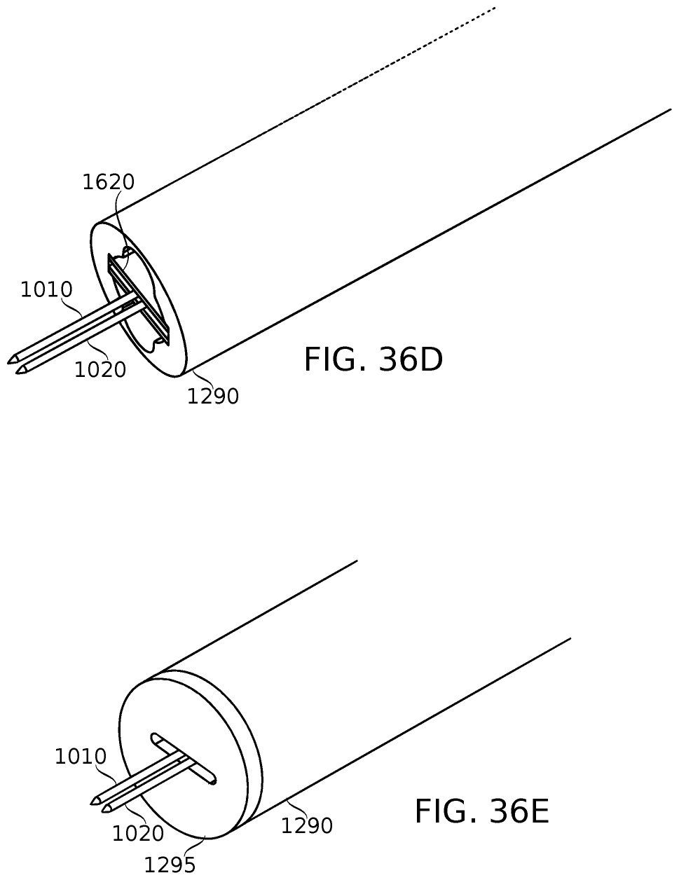

Reference is now made to FIGS. 36A-36E, which schematically illustrate construction of a mechanically actuated electrode assembly comprising two needle electrodes 1010, 1020 interconnected by a loop spring 1600, according to some embodiments of the present disclosure.

Regardless of the transition temperature of the material of loop spring 1600, a loop spring 1600 can also be opened by mechanical force.

FIG. 36A shows elements of such a mechanism, which operates by movement of a wedge 1610 to force the aperture of loop spring 1600 open. The mechanism comprises two side plates 1615 on either side of a wedge 1610. The side plates 1615 are coupled to either side of loop spring 1600, so that when they are separated, the aperture of loop spring 1600 is also opened, resulting the lateral separation of electrodes 1010, 1020.

With wedge 1610 in its withdrawn position (FIG. 36A), side plates 1615 are free to be pulled into their laterally collapsed state by normally closed loop spring 1600. To the elements of FIG. 36A, FIG. 36B adds cover plates 1630 which help maintain wedge 1610 in alignment with side plates 1615. FIG. 36C adds shaft 1190 which rigidly attaches to wedge 1610. FIG. 36D adds outer tube 1290, with a cutaway showing internal details such as how plates 1620 are positioned within outer tube 1290 (to which they are rigidly attached). Shaft 1190 can be longitudinally translated through outer tube 1290, moving wedge 1610 longitudinally.

FIG. 36E shows the same view as FIG. 36D, without the cutaway, and with the addition of an end cover 1295 comprising a slot 1296 along which electrodes 1010, 1020 move when they are displaced by the opening of loop spring 600.

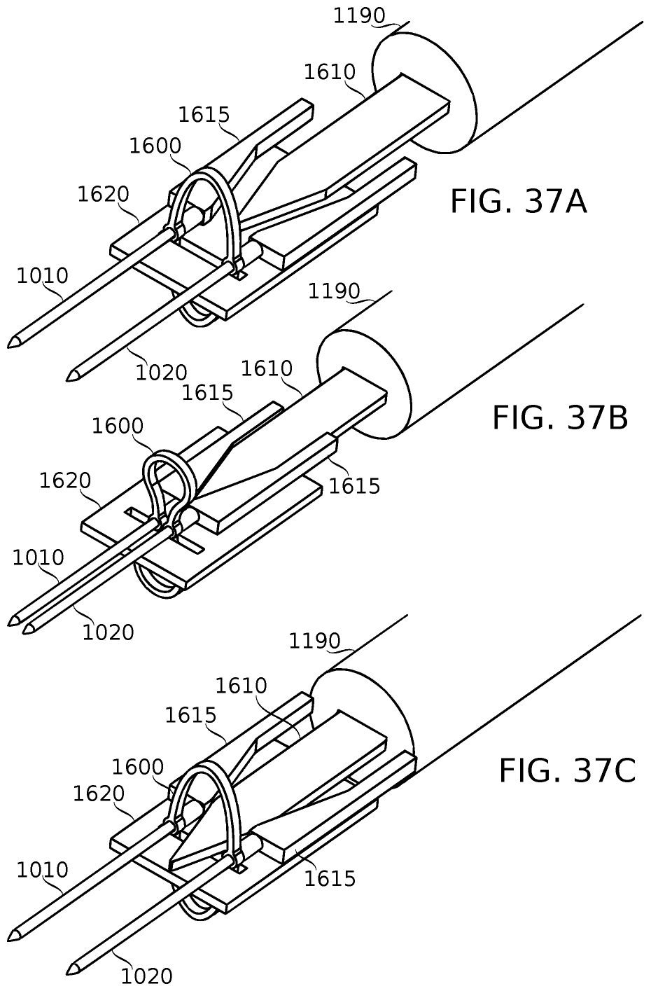

Reference is now made to FIGS. 37A-37C, which schematically illustrate operation of the mechanically actuated electrode assembly of FIGS. 36A-36E, according to some embodiments of the present disclosure. In each of these figures, indication of outer tube 1290 is suppressed, along with one of the cover plates 1620.

Comparing FIGS. 37A and 37B, it may be seen that plates 1615 are free to slide as loop 1600 transitions between a collapsed and an open configuration. This may happen, for example, as a result of a temperature-induced phase transition, e.g., as described in relation to FIGS. 35A-35B.

FIG. 37C shows wedge 1610 in a longitudinally advanced position, at which it forces plates 1615 apart. This in turn results in forcing loop spring 1600 into its open position, with electrodes 1610, 1620 laterally separated to a wider distance than when spring 1600 is in its closed position.

This be understood as a “reset” mechanism that allows returning loop 1600 to its open state again after it cools below its transition temperature. Once the device is reset, wedge 1600 can be retracted again. Loop spring 1600 remains in its open position until heated again. This has the potential advantage of allowing actuation of shaft 1190 as a momentary “pushbutton” switch—it can immediately spring back to the configuration of FIG. 37A, and loop spring 1600 will be reset.

Alternatively, the device of FIGS. 36A-37C can be provided with loop spring 1600 comprised of a superelastic material having a transition temperature below body temperature (e.g., below 37° C.). This allows it to remain fully elastic at all times. In such embodiments, opening or closing of the aperture of loop spring 1600 will be set by the position of width 1610 relative to plates 1615, regardless of operating temperature.

### General

As used herein with reference to quantity or value, the term “about” means “within ±10% of”.

The terms “comprises”, “comprising”, “includes”, “including”, “having” and their conjugates mean: “including but not limited to”.

The term “consisting of” means: “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

The words “example” and “exemplary” are used herein to mean “serving as an example, instance or illustration”. Any embodiment described as an “example” or “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the present disclosure may include a plurality of “optional” features except insofar as such features conflict.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

Throughout this application, embodiments may be presented with reference to a range format. It should be understood that the description in range format is merely for convenience and brevity and should not be construed as an inflexible limitation on the scope of descriptions of the present disclosure. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Although descriptions of the present disclosure are provided in conjunction with specific embodiments, it is evident that many alternatives, modifications and variations will be apparent to those skilled in the art. Accordingly, it is intended to embrace all such alternatives, modifications and variations that fall within the spirit and broad scope of the appended claims.

It is appreciated that certain features which are, for clarity, described in the present disclosure in the context of separate embodiments, may also be provided in combination in a single embodiment. Conversely, various features, which are, for brevity, described in the context of a single embodiment, may also be provided separately or in any suitable subcombination or as suitable in any other described embodiment of the present disclosure. Certain features described in the context of various embodiments are not to be considered essential features of those embodiments, unless the embodiment is inoperative without those elements.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present disclosure. To the extent that section headings are used, they should not be construed as necessarily limiting. In addition, any priority document(s) of this application is/are hereby incorporated herein by reference in its/their entirety.

1. A method of performing a cardiac annuloplasty procedure, comprising:
mechanically deforming tissue of a perimeter of an annulus of a heart valve; and
delivering energy to the mechanically deformed tissue, in an amount sufficient to structurally disrupt the mechanically deformed tissue and induce shrinkage of the valve annulus perimeter to reduce regurgitation through the heart valve;
wherein mechanical deforming comprises:
piercing the tissue with at least one sharpened element, and
exerting torsion on the tissue using the at least one sharpened element.

2-5. (canceled)

6. The method of claim 1, wherein the at least one sharpened element is used to deliver the structurally disruptive energy to the tissue.

7. The method of claim 6, wherein the at least one sharpened element delivers the structurally disruptive energy to the tissue by operating as an electrode.

8-10. (canceled)

11. The method of claim 1, wherein the mechanical deforming comprises compression including pinching the tissue between a plurality of the at least one sharpened element.

12-16. (canceled)

17. The method of claim 1, wherein the tissue-ablating energy is provided by at least one of the group consisting of:
radiofrequency energy;
focused ultrasound energy;
cryogenic cooling.

18. The method of claim 1, wherein the tissue ablated comprises at least one of the group consisting of:
fibrous tissue of the valve annulus; and
tissue of the heart wall adjacent to the fibrous tissue of the valve annulus.

19-23. (canceled)

24. A method of performing cardiac annuloplasty, comprising:
piercing tissue along a perimeter of a heart valve with at least one electrode;
applying mechanical force to the at least one electrode to deform the pierced tissue and reduce the perimeter of the heart valve annulus;
delivering tissue-ablating energy through the electrode, thereby inducing plastic deformation of the deformed tissue; and
releasing the mechanical force, leaving the heart valve annulus with a reduced perimeter;
wherein the applying mechanical force comprises placing torsion on the pierced tissue.

25. (canceled)

26. The method of claim 24, wherein the applying mechanical force comprises compressing the pierced tissue.

27. The method of claim 24, wherein the tissue-ablating energy is radiofrequency energy.

28. (canceled)

29. The method of claim 24, wherein the reduced perimeter draws leaflets of the heart valve into positions that reduce regurgitation of the valve.

30-31. (canceled)

32. A device for annuloplasty treatment, comprising:
a catheter, sized for transvascular insertion to a heart chamber from a percutaneous incision to reach a heart valve annulus thereof;
at least one tissue penetrating element at a distal end of the catheter;
wherein the at least one penetrating element both moves relative to a body of the catheter, and acts to deliver tissue disrupting energy to penetrated tissue of the heart valve annulus; and
wherein the at least one penetrating element has a non-circular cross-section that engages with and induces torsion in tissue to which it is inserted, upon receiving torque exerted through the catheter.

33. The device of claim 32, wherein the at least one penetrating element comprises a plurality of penetrating elements, adjustable in their relative distance while inserted to tissue of the heart valve annulus.

34. The device of claim 32, wherein each of the at least one tissue penetrating elements is an electrode electrically interconnected to a connection remaining outside the percutaneous incision when the catheter is inserted to the heart chamber.

35. The device of claim 33, wherein each of the plurality of penetrating elements operates as an ablation electrode.

36. The device of claim 33, wherein the penetrating elements are spaced to insert to the tissue at a relatively wider distance, and adjust to a narrower distance.

37-39. (canceled)

40. The device of claim 33, wherein the relative distance of the penetrating elements is adjusted by a temperature change of an actuator comprising a shape memory alloy.

41-45. (canceled)

46. The device of claim 32, comprising:
an inner component terminating distally in a tissue ablation segment, and housed within an outer tube;
the outer tube being sized for insertion to the heart chamber from within a guiding catheter;
wherein the outer tube is provided with a predetermined distal bend which it assumes when unconfined by the guiding catheter, and which straightens when the outer tube is withdrawn into the guiding catheter.

47. The device of claim 32, wherein the non-circular cross-section comprises a rectangular blade.

48. The device of claim 32, wherein the non-circular cross-section comprises three or more blades radiating from a central axis.

49. The method of claim 1, wherein the at least one sharpened elements comprises a plurality of sharpened elements, and the tissue is compressed by the torsion without change in distance between any of the plurality of sharpened elements.

50. The method of claim 1, comprising measuring impedance using the at least one sharpened element, and adjusting one or more of the delivery energy and operations to perform the piercing, using the measured impedance.

51. The method of claim 1, wherein the piercing the tissue comprises:
placing a casing containing the at least one sharpened element in contact with the tissue; and
extending the at least one sharpened element out of the casing and into the tissue.

52. The method of claim 51, wherein the at least one sharpened element comprises a plurality of sharpened elements extending from the casing parallel to each other.

53. The method of claim 51, wherein a depth of the piercing of the tissue is limited by a distal surface of the casing.

54. The method of claim 1, wherein the energy delivered comprises at least 112 Joules delivered over a period of at least 12 seconds.
